(19) United States
(12) Patent Application Publication
(10) Pub. No.: US 2005/0220854 A1
(43) Pub. Date: Oct. 6, 2005

(54) Apparatus and method for transdermal delivery of influenza vaccine

(76) Inventors: Yuh-Fun Maa, Millbrae, CA (US); Scott Sellers, San Mateo, CA (US); James Matriano, Mountain View, CA (US); Asha Ramdas, Sunnyvale, CA (US)

Correspondence Address: Ralph C. Francis;Francis Law Group, 1942 Embarcadero, Oakland, CA, 94606, US

(21) Appl. No.: 11/084,631

(22) Filed: Mar. 18, 2005

Related U.S. Application Data
(60) Provisional application No. 60/559,153, filed on Apr. 1, 2004.

(51) Int. Cl. A61K 39/12; A61K 39/02; A61K 9/70; A61M 31/00

(52) U.S. Cl. 424/449; 424/204.1; 424/234.1; 604/500

(57) ABSTRACT

An apparatus and method for transdermally delivering an immunologically active agent comprising a delivery system having a microprojection member (or system) that includes a plurality of microprojections (or array thereof) that are adapted to pierce through the stratum corneum into the underlying epidermis layer, or epidermis and dermis layers, the microprojection member having a biocompatible coating disposed thereon that includes the immunologically active agent. Preferably, the biocompatible coating is formed from a vaccine coating formulation.

### CROSS-REFERENCE TO RELATED APPLICATIONS

This application claims the benefit of U.S Provisional Application No. 60/559,153, filed Apr. 1, 2004.

### FIELD OF THE PRESENT INVENTION

The present invention relates generally to transdermal agent delivery systems and methods. More particularly, the invention relates to an apparatus, method and formulation for transdermal delivery of an influenza vaccine.

### BACKGROUND OF THE INVENTION

Active agents (or drug) are most conventionally administered either orally or by injection. Unfortunately, many active agent are completely ineffective or have radically reduced efficacy when orally administered, since they either are not absorbed or are adversely affected before entering the bloodstream and thus do not possess the desired activity. On the other hand, the direct injection of the agent into the bloodstream, while assuring no modification of the agent during administration, is a difficult, inconvenient, painful and uncomfortable procedure which sometimes results in poor patient compliance.

Hence, in principle, transdermal delivery provides for a method of administering active agents that would otherwise need to be delivered via hypodermic injection or intravenous infusion. The word “transdermal”, as used herein, is generic term that refers to delivery of an active agent (e.g., a therapeutic agent, such as a drug or an immunologically active agent, such as a vaccine) through the skin to the local tissue or systemic circulatory system without substantial cutting or penetration of the skin, such as cutting with a surgical knife or piercing the skin with a hypodermic needle. Transdermal agent delivery includes delivery via passive diffusion as well as delivery based upon external energy sources, such as electricity (e.g., iontophoresis) and ultrasound (e.g., phonophoresis).

As is well known in the art, skin is not only a physical barrier that shields the body from external hazards, but is also an integral part of the immune system. The immune function of the skin arises from a collection of residential cellular and humeral constituents of the viable epidermis and dermis with both innate and acquired immune functions, collectively known as the skin immune system.

One of the most important components of the skin immune system are the Langerhan's cells (LC), which are specialized antigen presenting cells found in the viable epidermis. LC's form a semi-continuous network in the viable epidermis due to the extensive branching of their dendrites between the surrounding cells. The normal function of the LC's is to detect, capture and present antigens to evoke an immune response to invading pathogens. LC's perform his function by internalizing epicutaneous antigens, trafficking to regional skin-draining lymph nodes, and presenting processed antigens to T cells.

The effectiveness of the skin immune system is responsible for the success and safety of vaccination strategies that have been targeted to the skin. Vaccination with a live-attenuated smallpox vaccine by skin scarification has successfully led to global eradication of the deadly small pox disease. Intradermal injection using ⅕ to 1/10 of the standard IM doses of various vaccines has been effective in inducing immune responses with a number of vaccines while a low-dose rabies vaccine has been commercially licensed for intradermal application.

As an alternative, transdermal delivery provides for a method of administering biologically active agents, particularly vaccines, that would otherwise need to be delivered via hypodermic injection, intravenous infusion or orally. Transdermal vaccine delivery offers improvements in both of these areas. Transdermal delivery when compared to oral delivery avoids the harsh environment of the digestive tract, bypasses gastrointestinal drug metabolism, reduces first-pass effects, and avoids the possible deactivation by digestive and liver enzymes. Conversely, the digestive tract is not subjected to the vaccine during transdermal administration.

Passive transdermal agent delivery systems, which are more common, typically include a drug reservoir that contains a high concentration of an active agent. The reservoir is adapted to contact the skin, which enables the agent to diffuse through the skin and into the body tissues or bloodstream of a patient.

One common method of increasing the passive transdermal diffusional agent flux involves pre-treating the skin with, or co-delivering with the agent, a skin permeation enhancer. A permeation enhancer, when applied to a body surface through which the agent is delivered, enhances the flux of the agent therethrough. However, the efficacy of these methods in enhancing transdermal protein flux has been limited, at least for the larger proteins, due to their size.

There also have been many techniques and systems developed to mechanically penetrate or disrupt the outermost skin layers thereby creating pathways into the skin in order to enhance the amount of agent being transdermally delivered. Early vaccination devices known as scarifiers generally include a plurality of tines or needles that were applied to the skin to and scratch or make small cuts in the area of application. The vaccine was applied either topically on the skin, such as disclosed in U.S. Pat. No. 5,487,726, or as a wetted liquid applied to the scarifier tines, such as, disclosed in U.S. Pat. Nos. 4,453,926, 4,109,655, and 3,136,314.

Scarifiers have been suggested for intradermal vaccine delivery, in part, because only very small amounts of the vaccine need to be delivered into the skin to be effective in immunizing the patient. Further, the amount of vaccine delivered is not particularly critical since an excess amount also achieves satisfactory immunization.

However, a serious disadvantage in using a scarifier to deliver an active agent, such as a vaccine, is the difficulty in determining the transdermal agent flux and the resulting dosage delivered. Also, due to the elastic, deforming and resilient nature of skin to deflect and resist puncturing, the tiny piercing elements often do not uniformly penetrate the skin and/or are wiped free of a liquid coating of an agent upon skin penetration.

Additionally, due to the self-healing process of the skin, the punctures or slits made in the skin tend to close up after removal of the piercing elements from the stratum corneum. Thus, the elastic nature of the skin acts to remove the active agent liquid coating that has been applied to the tiny piercing elements upon penetration of these elements into the skin. Furthermore, the tiny slits formed by the piercing elements heal quickly after removal of the device, thus limiting the passage of the liquid agent solution through the passageways created by the piercing elements and in turn limiting the transdermal flux of such devices.

Other systems and apparatus that employ tiny skin piercing elements to enhance transdermal agent delivery are disclosed in U.S. Pat. Nos. 5,879,326, 3,814,097, 5,279,54, 5,250,023, 3,964,482, Reissue No. 25,637, and PCT Publication Nos. WO 96/37155, WO 96/37256, WO 96/17648, WO 97/03718, WO 98/11937, WO 98/00193, WO 97/48440, WO 97/48441, WO 97/48442, WO 98/00193, WO 99/64580, WO 98/28037, WO 98/29298, and WO 98/29365; all incorporated herein by reference in their entirety.

The disclosed systems and apparatus employ piercing elements of various shapes and sizes to pierce the outermost layer (i.e., the stratum corneum) of the skin. The piercing elements disclosed in these references generally extend perpendicularly from a thin, flat member, such as a pad or sheet. The piercing elements in some of these devices are extremely small, some having a microprojection length of only about 25-400 microns and a microprojection thickness of only about 5-50 microns. These tiny piercing/cutting elements make correspondingly small microslits/microcuts in the stratum corneum for enhancing transdermal agent delivery therethrough.

The disclosed systems further typically include a reservoir for holding the agent and also a delivery system to transfer the agent from the reservoir through the stratum corneum, such as by hollow tines of the device itself. One example of such a device is disclosed in WO 93/17754, which has a liquid agent reservoir. The reservoir must, however, be pressurized to force the liquid agent through the tiny tubular elements and into the skin. Disadvantages of such devices include the added complication and expense for adding a pressurizable liquid reservoir and complications due to the presence of a pressure-driven delivery system.

As disclosed in U.S. patent application Ser. No. 10/045,842, which is fully incorporated by reference herein, it is also possible to have the active agent that is to be delivered coated on the microprojections instead of contained in a physical reservoir. This eliminates the necessity of a separate physical reservoir and developing an agent formulation or composition specifically for the reservoir.

A drawback of the coated microprojection systems is, however, that the maximum amount of delivered active agent, and in particular, immunologically active agents, is limited, since the ability of the microprojections (and arrays thereof to penetrate the stratum corneum is reduced as the coating thickness increases. Further, to effectively penetrate the stratum corneum with microprojections having a thick coating disposed thereon, the impact energy of the applicator must be increased, which causes intolerable sensations upon impact.

It would therefore be desirable to provide a high concentration immunologically active agent, and in particular, a liquid influenza vaccine that can be readily administered in an immunologically (or biologically) effective amount via coated microprojections.

Accordingly, it is an object of the present invention to provide an apparatus and method for transdermal delivery of an immunologically active agent that substantially reduces or eliminates the drawbacks and disadvantages associated with prior art immunologically active agent delivery methods and systems.

It is another object of the present invention to provide an apparatus and method for transdermal delivery of influenza vaccine that includes microprojections coated with a biocompatible coating having the influenza vaccine disposed therein.

It is another object of the present invention to provide an apparatus and method for transdermal delivery of influenza vaccine that includes a microprojection member having a plurality of microprojections, wherein the microprojections are coated with an influenza vaccine coating formulation.

It is yet another object of the present invention to provide an influenza vaccine that can be readily administered in an immunologically effective amount via a coated microprojection system.

It is another object of the present invention to provide an influenza vaccine that is substantially preservative free.

It is yet another object of the present invention to provide an influenza vaccine that has an enhanced shelf life.

### SUMMARY OF THE INVENTION

In accordance with the above objects and those that will be mentioned and will become apparent below, the apparatus and method for transdermally delivering an immunologically active agent in accordance with this invention generally comprises a delivery system having a microprojection member (or system) that includes a plurality of microprojections (or array thereof) that are adapted to pierce through the stratum corneum into the underlying epidermis layer, or epidermis and dermis layers, the microprojection member having a biocompatible coating disposed thereon that includes the immunologically active agent. In a preferred embodiment of the invention, the biocompatible coating is formed from an immunologically active agent coating formulation.

In a preferred embodiment of the invention, the immunologically active agent comprises an influenza vaccine.

In alternative embodiments of the invention, the immunologically active agent comprises an antigenic agent or vaccine selected from the group consisting of viruses and bacteria, protein-based vaccines, polysaccharide-based vaccine, and nucleic acid-based vaccines.

Suitable antigenic agents include, without limitation, antigens in the form of proteins, polysaccharide conjugates, oligosaccharides, and lipoproteins. These subunit vaccines in include Bordetella pertussis (recombinant PT accince—acellular), Clostridium tetani (purified, recombinant), Corynebacterium diphtheriae (purified, recombinant), Cytomegalovirus (glycoprotein subunit), Group A streptococcus (glycoprotein subunit, glycoconjugate Group A polysaccharide with tetanus toxoid, M protein/peptides linked to toxing subunit carriers, M protein, multivalent type-specific epitopes, cysteine protease, C5a peptidase), Hepatitis B virus (recombinant Pre S1, Pre-S2, S, recombinant core protein), Hepatitis C virus (recombinant—expressed surface proteins and epitopes), Human papillomavirus (Capsid protein, TA-GN recombinant protein L2 and E7 [from HPV-6], MEDI-501 recombinant VLP L1 from HPV-11, Quadrivalent recombinant BLP L1 [from HPV-6], HPV-11, HPV-16, and HPV-18, LAMP-E7 [from HPV-16]), Legionella pneumophila (purified bacterial survace protein), Neisseria meningitides (glycoconjugate with tetanus toxoid), Pseudomonas aeruginosa (synthetic peptides), Rubella virus (synthetic peptide), Streptococcus pneumoniae (glyconconjugate [1, 4, 5, 6B, 9N, 14, 18C, 19V, 23F] conjugated to meningococcal B OMP, glycoconjugate [4, 6B, 9V, 14, 18C, 19F, 23F] conjugated to CRM197, glycoconjugate [1, 4, 5, 6B, 9V, 14, 18C, 19F, 23F] conjugated to CRM1970, Treponema pallidum (surface lipoproteins), Varicella zoster virus (subunit, glycoproteins), and Vibrio cholerae (conjugate lipopolysaccharide).

Whole virus or bacteria include, without limitation, weakened or killed viruses, such as cytomegalo virus, hepatitis B virus, hepatitis C virus, human papillomavirus, rubella virus, and varicella zoster, weakened or killed bacteria, such as bordetella pertussis, clostridium tetani, corynebacterium diphtheriae, group A streptococcus, legionella pneumophila, neisseria meningitdis, pseudomonas aeruginosa, streptococcus pneumoniae, treponema pallidum, and vibrio cholerae, and mixtures thereof.

Additional commercially available vaccines, which contain antigenic agents, include, without limitation, flu vaccines, Lyme disease vaccine, rabies vaccine, measles vaccine, mumps vaccine, chicken pox vaccine, small pox vaccine, hepatitis vaccine, pertussis vaccine, and diphtheria vaccine.

Vaccines comprising nucleic acids include, without limitation, single-stranded and double-stranded nucleic acids, such as, for example, supercoiled plasmid DNA; linear plasmid DNA; cosmids; bacterial artificial chromosomes (BACs); yeast artificial chromosomes (YACs); mammalian artificial chromosomes; and RNA molecules, such as, for example, mRNA. The nucleic acid can also be coupled with a proteinaceous agent or can include one or more chemical modifications, such as, for example, phosphorothioate moieties.

Suitable immune response augmenting adjuvants which, together with the vaccine antigen, can comprise the vaccine include aluminum phosphate gel; aluminum hydroxide; algal glucan: β-glucan; cholera toxin B subunit; CRL1005: ABA block polymer with mean values of x=8 and y=205; gamma inulin: linear (unbranched) β-D(2->1) polyfructofuranoxyl-β-D-glucose; Gerbu adjuvant: N-acetylglucosamine-(β 1-4)-N-acetylmuramyl-L-alanyl-D-glutamine (GMDP), dimethyl dioctadecylammonium chloride (DDA), zinc L-proline salt complex (Zn-Pro-8); Imiquimod (1-(2-methypropyl)-1H-imidazo[4,5-c]quinolin-4-amine; ImmTher™: N-acetylglucoaminyl-N-acetylmuramyl-L-Ala-D-isoGlu-L-Ala-glycerol dipalmitate; MTP-PE liposomes: C₅₉H₁₀₈N₆O₁₉PNa-3H₂O (MTP); Murametide: Nac-Mur-L-Ala-D-Gln-OCH₃; Pleuran: β-glucan; QS-21; S-28463: 4-amino-a, a-dimethyl-1H-imidazo[4,5-c]quinoline-1-ethanol; salvo peptide: VQGEESNDK•HCl (IL-1β163-171 peptide); and threonyl-MDP (Termurtide™): N-acetyl muramyl-L-threonyl-D-isoglutamine, and interleukine 18, IL-2 IL-1 2, L-15, Adjuvants also include DNA oligonucleotides, such as, for example, CpG containing oligonucleotides. In addition, nucleic acid sequences encoding for immuno-regulatory lymphokines such as IL-1 8, IL-2 IL-12, IL-15, IL-4, IL10, gamma interferon, and NF kappa B regulatory signaling proteins can be used.

In one embodiment of the invention, the microprojection member has a microprojection density of at least approximately 10 microprojections/cm², preferably, greater than approximately 100 microprojections/cm², and more preferably, in the range of approximately 200-3000 microprojections/cm². Further, each of the microprojections preferably has a length in the range of approximately 50 - 145 microns, and more preferably, in the range of approximately 70-140 microns.

In one embodiment, the microprojection member is constructed out of stainless steel, titanium, nickel titanium alloys, or similar biocompatible materials, such as polymeric materials.

In an alternative embodiment, the microprojection member is constructed out of a non-conductive material, such as a polymer. Alternatively, the microprojection member can be coated with a non-conductive material, such as Parylene®.

In one embodiment of the invention, the biocompatible coating has a thickness less than 100 microns. In a preferred embodiment, the biocompatible coating has a thickness in the range of approximately 2-50 microns.

The coating formulation applied to the microprojection member to form a solid biocompatible coating can comprise an aqueous or non-aqueous formulation that includes the immunologically active agent. In a preferred embodiment, the coating formulation comprises an aqueous formulation.

In one embodiment of the invention, the coating formulation includes at least one surfactant, which can be zwitterionic, amphoteric, cationic, anionic, or nonionic, Suitable surfactants include, without limitation, sodium lauroamphoacetate, sodium dodecyl sulfate (SDS), cetylpyridinium chloride (CPC), dodecyltrimethyl ammonium chloride (TMAC), benzalkonium, chloride, polysorbates, such as Tween 20 and Tween 80, other sorbitan derivatives, such as sorbitan laurate, and alkoxylated alcohols, such as laureth-4.

In a further embodiment of the invention, the coating formulation includes at least one polymeric material or polymer that has amphiphilic properties, which can comprise, without limitation, dextrans, hydroxyethyl starch (HES), cellulose derivatives, such as hydroxyethylcellulose (HEC), hydroxypropylmethylcellulose (HPMC), hydroxypropycellulose (HPC), methylcellulose (MC), hydroxyethylmethylcellulose (HEMC), or ethylhydroxy-ethylcellulose (EHEC), as well as pluronics.

In one embodiment of the invention, the concentration of the polymer presenting amphiphilic properties in the coating formulation is preferably in the range of approximately 0.001-70 wt. %, more preferably, in the range of approximately 0.01-50 wt. %, even more preferably, in the range of approximately 0.03-30 wt. % of the coating formulation.

In one embodiment of the invention, the concentration of the polymer presenting amphiphilic properties in the solid biocompatible coating is preferably in the range of approximately 0.002-99.9 wt. %, more preferably, in the range of approximately 0.1-60 wt. % of the solid biocompatible coating.

In another embodiment, the coating formulation includes a hydrophilic polymer selected from the following group: poly(vinyl alcohol), poly(ethylene oxide), poly(2-hydroxyethylmethacrylate), poly(n-vinyl pyrolidone), polyethylene glycol and mixtures thereof, and like polymers.

In a preferred embodiment, the concentration of the hydrophilic polymer in the coating formulation is preferably in the range of approximately 0.001-90 wt. %, more preferably, in the range of approximately 0.01-20 wt. %, even more preferably, in the range of approximately 0.03-10 wt. % of the coating formulation.

In a preferred embodiment, the concentration of the hydrophilic polymer in the solid biocompatible coating is in the range of approximately 0.002-99.9 wt. %, more preferably, in the range of approximately 0.1-20 wt. % of the coating formulation.

In another embodiment of the invention, the coating formulation includes a biocompatible carrier, which can comprise, without limitation, human albumin, bioengineered human albumin, polyglutamic acid, polyaspartic acid, polyhistidine, pentosan polysulfate, polyamino acids, sucrose, trehalose, melezitose, raffinose and stachyose.

Preferably, the concentration of the biocompatible carrier in the coating formulation is preferably in the range of approximately 0.001-90%, more preferably, in the range of approximately 2-70 wt. %, even more preferably, in the range of approximately 5-50 wt. % of the coating formulation.

Preferably, the concentration of the biocompatible carrier in the solid biocompatible coating is in the range of approximately 0.002-99.9 wt. %, more preferably, in the range of approximately 0.1-95 wt. % of the solid biocompatible formulation.

In a further embodiment, the coating formulation includes a stabilizing agent, which can comprise, without limitation, a non-reducing sugar, a polysaccharide, a reducing sugar, or a DNase inhibitor.

In another embodiment, the coating formulation includes a vasoconstrictor, which can comprise, without limitation, amidephrine, cafaminol, cyclopentamine, deoxyepinephrine, epinephrine, felypressin, indanazoline, metizoline, midodrine, naphazoline, nordefrin, octodrine, omipressin, oxymethazoline, phenylephrine, phenylethanolamine, phenylpropanolamine, propylhexedrine, pseudoephedrine, tetrahydrozoline, tramazoline, tuaminoheptane, tymazoline, vasopressin, xylometazoline and the mixtures thereof. The most preferred vasoconstrictors include epinephrine, naphazoline, tetrahydrozoline indanazoline, metizoline, tramazoline, tymazoline, oxymetazoline and xylometazoline.

The concentration of the vasoconstrictor, if employed, is preferably in the range of approximately 0.1 wt. % to 10 wt. % of the coating.

In yet another embodiment of the invention, the coating formulation includes at least one “pathway patency modulator”, which can comprise, without limitation, osmotic agents (e.g., sodium chloride), zwitterionic compounds (e.g., amino acids), and anti-inflammatory agents, such as betamethasone 21-phosphate disodium salt, triamcinolone acetonide 21-disodium phosphate, hydrocortamate hydrochloride, hydrocortisone 21-phosphate disodium salt, methylprednisolone 21-phosphate disodium salt, methylprednisolone 21-succinaate sodium salt, paramethasone disodium phosphate and prednisolone 21-succinate sodium salt, and anticoagulants, such as citric acid, citrate salts (e.g., sodium citrate), dextrin sulfate sodium, aspirin and EDTA.

Preferably, the coating formulations of the invention have a viscosity less than approximately 5 poise, more preferably, in the range of approximately 0.3-2.0 poise.

In accordance with one embodiment of the invention, the method for delivering an immunologically active agent comprises the following steps: (i) providing a microprojection member having a plurality of microprojections, (ii) providing a bulk vaccine, (iii) subjecting the bulk vaccine to tangential-flow filtration to provide a vaccine solution, (iv) adding at least one excipient (e.g., sucrose, trehalose or mannitol) to the vaccine solution, (v) freeze-drying the vaccine solution to form a vaccine product, (vi) reconstituting the vaccine product with a first solution (e.g., water) to form a vaccine coating formulation, (vii) coating the microprojection member with the vaccine coating formulation, and (viii) applying the coated microprojection member to the skin of a subject.

In one embodiment, the vaccine comprises an influenza vaccine. Preferably, the method comprises the step of delivering approximately 45 μg of hemagglutinin. More preferably, the step of delivering the vaccine comprises delivering at least approximately 70% of the vaccine to the APC-abundant epidermal layer.

In another embodiment, a method for formulating a vaccine solution of the invention comprises the following steps: (i) providing a bulk vaccine, (ii) subjecting the bulk vaccine to tangential-flow filtration to provide a vaccine solution, (iii) adding at least one excipient to the vaccine solution, (iv) freeze-drying the vaccine solution to form a vaccine product. In one embodiment, the vaccine product exhibits a concentration that is at least 500-fold more concentrated than the bulk vaccine. Preferably, the vaccine product maintains room temperature stability for at least approximately six months.

### BRIEF DESCRIPTION OF THE DRAWINGS

Further features and advantages will become apparent from the following and more particular description of the preferred embodiments of the invention, as illustrated in the accompanying drawings, and in which like referenced characters generally refer to the same parts or elements throughout the views, and in which:

FIG. 1 is an illustration of an influenza virus particle;

FIG. 2 is a perspective view of a portion of one embodiment of a microprojection member, according to the invention;

FIG. 3 is a perspective view of the microprojection member shown in FIG. 2 having a biocompatible coating deposited on the microprojections, according to the invention;

FIG. 4 is a sectioned side view of a microprojection member having an adhesive backing, according to the invention;

FIG. 5 is a perspective view of a portion of another embodiment of a microprojection member, according to the invention;

FIG. 6 is a sectioned side view of a retainer having a microprojection member disposed therein, according to the invention;

FIG. 7 is a perspective view of the retainer shown in FIG. 6;

FIG. 8 is a perspective view of an applicator and the retainer shown in FIG. 6;

FIG. 9 is a flow chart of a pre-formulation process, according to the invention;

FIG. 10 is a graphical illustration of absorbance versus pH illustrating pH effect on reducing solution turbidity, according to the invention;

FIG. 11 is a graphical illustration of viscosity versus rpm for the vaccines Fluzone® and Vaxigrip™;

FIG. 12 is a graphical illustration of viscosity versus temperature for a A/New Caledonia strain, having 15% HA purity at 22.5 mg/mL;

FIGS. 13A and 13B are graphical illustrations summarizing vaccine delivery for various microprojection array designs, according to the invention;

FIG. 14A is a graphical illustration of average anti-HA titer versus time for various doses of HA (A/Panama strain);

FIG. 14B is a graphical illustration of total A/Panama-specific IgG titers versus HI activity;

FIGS. 15A and 15B are bar charts of the immuongenicity of several formulations of HA (A/Panama strain), illustrating anti-A/Panama-specific IgG antibody and HI activity;

FIGS. 16A and 16B are bar charts of the immuongenicity of several formulations of HA (A/Panama strain) dry-coated onto microprojections, illustrating anti-HA antibody activity:HI activity at day 28 and day 49;

FIG. 17 is a series of bar charts of the immuongenicity of several formulations of trivalent HA (A/Panama, A/New Caledonia and B/Shangdong strains) dry-coated onto microprojections, illustrating HI activity;

FIG. 18 is a graphical illustration of HA amount versus time, illustrating stability profiles of several coating formulations stored at 40° C. for up to eight weeks, according to the invention;

FIGS. 19 and 20 are bar charts of two trivalent formulations, illustrating stability profiles of the formulations stored at 40° C. for up to three months and 5° C. and 40° C. for up to six months, according to the invention; and

FIG. 21 is a graphical illustration of SRID/BCA versus time, showing stability profiles of an A/New Caledonia strain formulated with sucrose and stored at 40° C. for up to eight weeks, according to the invention.

### DETAILED DESCRIPTION OF THE INVENTION

Before describing the present invention in detail, it is to be understood that this invention is not limited to particularly exemplified materials, formulations, methods or structures as such may, of course, vary. Thus, although a number of materials and methods similar or equivalent to those described herein can be used in the practice of the present invention, the preferred materials and methods are described herein.

It is also to be understood that the terminology used herein is for the purpose of describing particular embodiments of the invention only and is not intended to be limiting.

Unless defined otherwise, all technical and scientific terms used herein have the same meaning as commonly understood by one having ordinary skill in the art to which the invention pertains.

Further, all publications, patents and patent applications cited herein, whether supra or infra, are hereby incorporated by reference in their entirety.

Finally, as used in this specification and the appended claims, the singular forms “a, “an” and “the” include plural referents unless the content clearly dictates otherwise. Thus, for example, reference to “an immunologically active agent” includes two or more such agents; reference to “a microprojection” includes two or more such microprojections and the like.

### Definitions

The term “transdermal”, as used herein, means the delivery of an agent into and/or through the skin for local or systemic therapy.

The term “transdermal flux”, as used herein, means the rate of transdermal delivery.

The term “co-delivering”, as used herein, means that a supplemental agent(s) is administered transdermally either before the agent is delivered, before and during transdermal flux of the agent, during transdermal flux of the agent, during and after transdermal flux of the agent, and/or after transdermal flux of the agent. Additionally, two or more immunologically active agents may be formulated in the biocompatible coatings of the invention, resulting in co-delivery of different immunologically active agents.

The term “biologically active agent”, as used herein, refers to a composition of matter or mixture containing an active agent or drug, which is pharmacologically effective when administered in a therapeutically effective amount. Examples of such active agents include, without limitation, small molecular weight compounds, polypeptides, proteins, oligonucleotides, nucleic acids and polysaccharides.

The term “immunologically active agent”, as used herein, refers to a composition of matter or mixture containing an antigenic agent and/or a “vaccine” from any and all sources, which is capable of triggering a beneficial immune response when administered in an immunologically effective amount. A specific example of an immunologically active agent is an influenza vaccine.

Further examples of immunologically active agents include, without limitation, viruses and bacteria, protein-based vaccines, polysaccharide-based vaccine, and nucleic acid-based vaccines.

Suitable immunologically active agents include, without limitation, antigens in the form of proteins, polysaccharide conjugates, oligosaccharides, and lipoproteins. These subunit vaccines in include Bordetella pertussis (recombinant PT accince—acellular), Clostridium tetani (purified, recombinant), Corynebacterium diphtheriae (purified, recombinant), Cytomegalovirus (glycoprotein subunit), Group A streptococcus (glycoprotein subunit, glycoconjugate Group A polysaccharide with tetanus toxoid, M protein/peptides linked to toxing subunit carriers, M protein, multivalent type-specific epitopes, cysteine protease, C5a peptidase), Hepatitis B virus (recombinant Pre SI, Pre-S2, S, recombinant core protein), Hepatitis C virus (recombinant - expressed surface proteins and epitopes), Human papillomavirus (Capsid protein, TA-GN recombinant protein L2 and E7 [from HPV-6], MEDI-501 recombinant VLP LI from HPV-1 1, Quadrivalent recombinant BLP L1 [from HPV-6], HPV-11, HPV-16, and HPV-18, LAMP-E7 [from HPV-16]), Legionella pneumophila (purified bacterial surface protein), Neisseria meningitides (glycoconjugate with tetanus toxoid), Pseudomonas aeruginosa (synthetic peptides), Rubella virus (synthetic peptide), Streptococcus pneumoniae (glyconconjugate [1, 4, 5, 6B, 9N, 14, 18C, 19V, 23F] onjugated to meningococcal B OMP, glycoconjugate [4, 6B, 9V, 14, 18C, 19F, 23F] conjugated to CRM197, glycoconjugate [1, 4, 5, 6B, 9V, 14, 18C, 19F, 23F] conjugated to CRM1970, Treponema pallidum (surface lipoproteins), Varicella zoster virus (subunit, glycoproteins), and Vibrio cholerae (conjugate lipopolysaccharide).

Whole virus or bacteria include, without limitation, weakened or killed viruses, such as cytomegalo virus, hepatitis B virus, hepatitis C virus, human papillomavirus, rubella virus, and varicella zoster, weakened or killed bacteria, such as bordetella pertussis, clostridium tetani, corynebacterium diphtheriae, group A streptococcus, legionella pneumophila, neisseria meningitis, pseudomonas aeruginosa, streptococcus pneumoniae, treponema pallidum, and vibrio cholerae, and mixtures thereof.

A number of commercially available vaccines, which contain antigenic agents also have utility with the present invention, include, without limitation, flu vaccines, Lyme disease vaccine, rabies vaccine, measles vaccine, mumps vaccine, chicken pox vaccine, small pox vaccine, hepatitis vaccine, pertussis vaccine, and diphtheria vaccine.

Vaccines comprising nucleic acids that can also be delivered according to the methods of the invention, include, without limitation, single-stranded and double-stranded nucleic acids, such as, for example, supercoiled plasmid DNA; linear plasmid DNA; cosmids; bacterial artificial chromosomes (BACs); yeast artificial chromosomes (YACs); mammalian artificial chromosomes; and RNA molecules, such as, for example, mRNA. The size of the nucleic acid can be up to thousands of kilobases. The nucleic acid can also be coupled with a proteinaceous agent or can include one or more chemical modifications, such as, for example, phosphorothioate moieties.

Suitable immune response augmenting adjuvants which, together with the vaccine antigen, can comprise the vaccine include, without limitation, aluminum phosphate gel; aluminum hydroxide; algal glucan: β-glucan; cholera toxin B subunit; CRL1005: ABA block polymer with mean values of x=8 and y=205; gamma inulin: linear (unbranched) β-D(2->1) polyfructofuranoxyl-α-D-glucose; Gerbu adjuvant: N-acetylglucosamine-(β 14)-N-acetylmuramyl-L-alanyl-D-glutamine (GMDP), dimethyl dioctadecylammonium chloride (DDA), zinc L-proline salt complex (Zn-Pro-8); Imiquimod (1-(2-methypropyl)-1H-imidazo[4,5-c]quinolin-4-amine; ImmTher™: N-acetylglucoaminyl-N-acetylmuramyl-L-Ala-D-isoGlu-L-Ala-glycerol dipalmitate; MTP-PE liposomes: C₅₉H₁₀₈N₆O₁₉PNa—3H₂O (MTP); Murametide: Nac-Mur-L-Ala-D-Gln-OCH₃; Pleuran: β-glucan; QS-21; S-28463: 4-amino-a, a-dimethyl-1H-imidazo[4,5-c]quinoline-1-ethanol; salvo peptide: VQGEESNDK•HCl (IL-1β 163-171 peptide); and threonyl-MDP (Termurtide™): N-acetyl muramyl-L-threonyl-D-isoglutamine, and interleukine 18, IL-2 IL-1 2, IL- 15, Adjuvants also include DNA oligonucleotides, such as, for example, CpG containing oligonucleotides. In addition, nucleic acid sequences encoding for immuno-regulatory lymphokines such as IL-1 8, IL-2 IL-1 2, IL-1 5, IL-4, IL10, gamma interferon, and NF kappa B regulatory signaling proteins can be used.

The term “biologically effective amount” or “biologically effective rate”, as used herein, refers to the amount or rate of the immunologically active agent needed to stimulate or initiate the desired immunologic, often beneficial result. The amount of the immunologically active agent employed in the coatings of the invention will be that amount necessary to deliver an amount of the immunologically active agent needed to achieve the desired immunological result. In practice, this will vary widely depending upon the particular immunologically active agent being delivered, the site of delivery, and the dissolution and release kinetics for delivery of the immunologically active agent into skin tissues.

As will be appreciated by one having ordinary skill in the art, the dose of the immunologically active agent that is delivered can also be varied or manipulated by altering the microprojection array (or patch) size, density, etc.

The term “coating formulation”, as used herein, is meant to mean and include a freely flowing composition or mixture that is employed to coat the microprojections and/or arrays thereof.

The term “biocompatible coating” and “solid coating”, as used herein, is meant to mean and include a “coating formulation” in a substantially solid state.

The term “microprojections”, as used herein, refers to piercing elements which are adapted to pierce or cut through the stratum corneum into the underlying epidermis layer, or epidermis and dermis layers, of the skin of a living animal, particularly a mammal and more particularly a human.

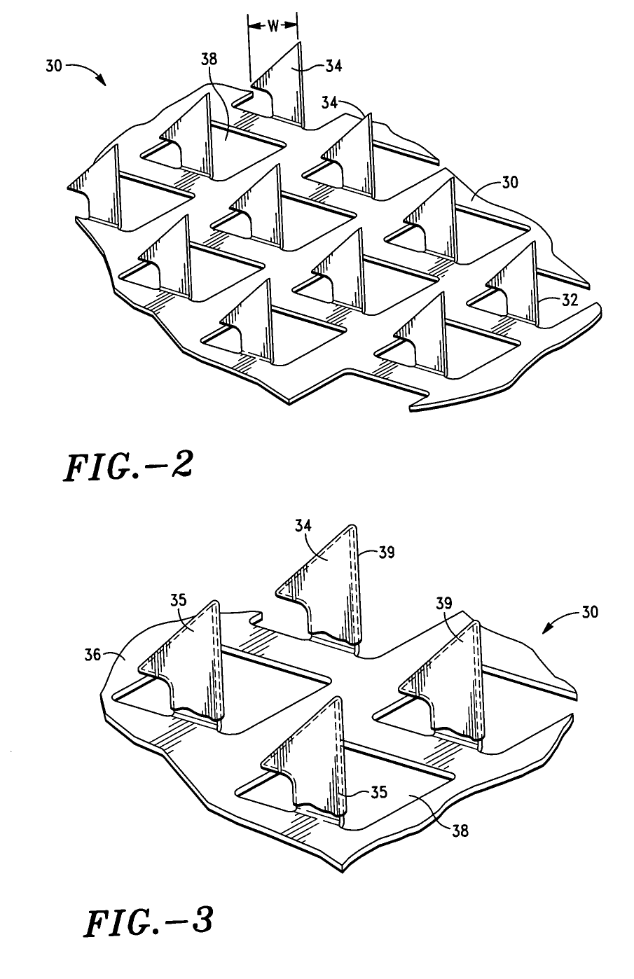

The term “microprojection member”, as used herein, generally connotes a microprojection array comprising a plurality of microprojections arranged in an array for piercing the stratum corneum. The microprojection member can be formed by etching or punching a plurality of microprojections from a thin sheet and folding or bending the microprojections out of the plane of the sheet to form a configuration, such as that shown in FIG. 2. The microprojection member can also be formed in other known manners, such as by forming one or more strips having microprojections along an edge of each of the strip(s) as disclosed in U.S. Pat. No. 6,050,988, which is hereby incorporated by reference in its entirety.

In one embodiment, the microprojection member has an array with a microprojection density of at least approximately 10 microprojections/cm², preferably, at least approximately 100 microprojections/cm², and more preferably, in the range of approximately 200-3000 microprojections/cm².

As indicated above, the present invention comprises an apparatus and method for transdermal delivery of an immunologically active agent that includes a microprojection member (or system) having a plurality of microprojections (or array thereof) that are adapted to pierce through the stratum corneum into the underlying epidermis layer, or epidermis and dermis layers, the microprojection member having a biocompatible coating disposed thereon that includes the immunologically active agent.

In a preferred embodiment of the invention, the immunologically active agent comprises an influenza vaccine, more preferably, a trivalent influenza vaccine. According to the invention, upon piercing the stratum corneum layer of the skin, the biocompatible coating is dissolved by body fluid (intracellular fluids and extracellular fluids such as interstitial fluid) and the influenza vaccine is released into the skin (i.e., bolus delivery) for systemic therapy.

According to the invention, the kinetics of the coating dissolution and release will depend on many factors, including the nature of the immunologically active agent, the coating process, the coating thickness and the coating composition (e.g., the presence of coating formulation additives). Depending on the release kinetics profile, it may be necessary to maintain the coated microprojections in piercing relation with the skin for extended periods of time. This can be accomplished by anchoring the microprojection member to the skin using adhesives or by using anchored microprojections, such as shown in FIG. 5 and described in WO 97/48440, which is incorporated by reference herein in its entirety.

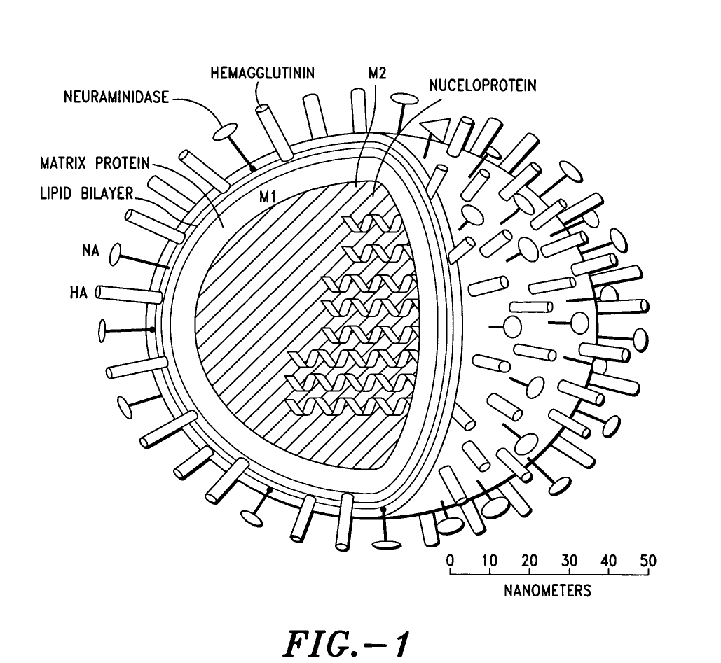

As is well known in the art, the influenza virus particle consists of many protein components with hemagglutinin (HA) as the primary surface antigen responsible for the induction of protective anti-HA antibodies in humans. An illustration of an influenza particle is shown in FIG. 1.

Immunologically, influenza A viruses are classified into subtypes on the basis of two surface antigens: HA and neuraminidase (NA). Immunity to these antigens, especially to the hemagglutinin, reduces the likelihood of infection of infection and lessens the severity of the disease if infection occurs.

The antigenic characteristics of circulating strains provide the basis for selecting the virus strains included in each year's vaccine. Every year, the influenza vaccine contains three virus strains (usually two type A and one B) that represent the influenza viruses that are likely to circulate worldwide in the coming winter. Influenza A and B can be distinguished by differences in their nucleoproteins and matrix proteins. Type A is the most common strain and is responsible for the major human pandemics. The HA content of each strain in the trivalent vaccine is typically set at 15 μg for a single human dose, i.e., 45 μg total HA.

As discussed in detail herein, by virtue of the unique-pre-formulation process, a full human dose of the influenza vaccine, i.e., 45 μg of hemagglutinin, can be transdermally delivered to the APC-abundant epidermal layer, the most immuno-competent component of the skin, via a coated microprojection array, wherein at least 70% of the influenza vaccine is delivered to the noted epidermal layer. More importantly, the antigen remains immunogenic in the skin to elicit strong antibody and sero-protective immune responses. Further, the dry coated vaccine formulation is substantially preservative-free and can maintain at least a six-month room temperature stability.

Referring now to FIG. 2, there is shown one embodiment of a microprojection member 30 for use with the present invention. As illustrated in FIG. 2, the microprojection member 30 includes a microprojection array 32 having a plurality of microprojections 34. The microprojections 34 preferably extend at substantially a 90° angle from the sheet 36, which in the noted embodiment includes openings 38.

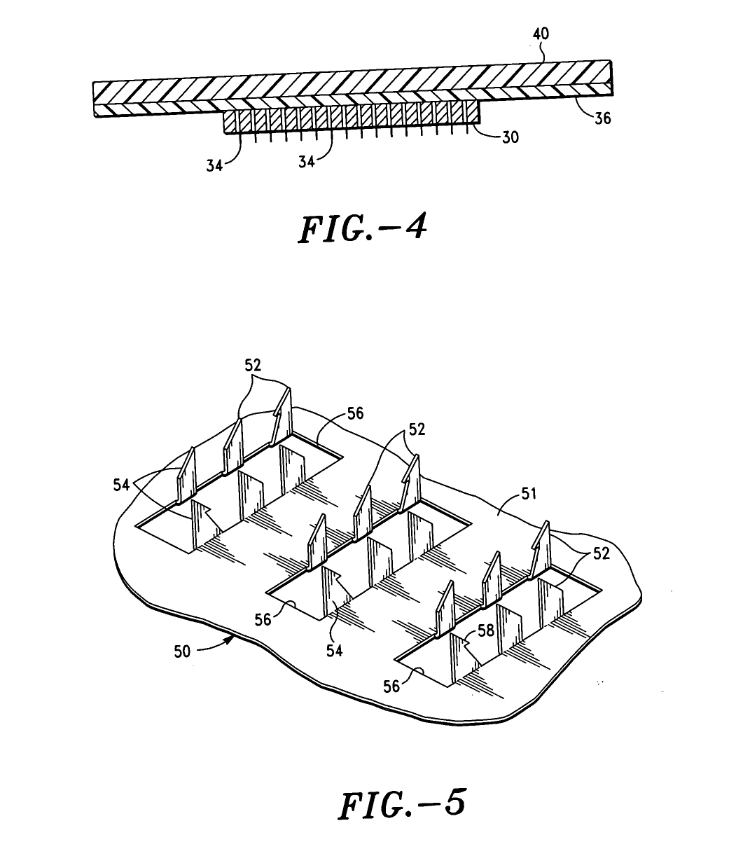

According to the invention, the sheet 36 may be incorporated into a delivery patch, including a backing 40 for the sheet 36, and may additionally include an adhesive strip (not shown) for adhering the patch to the skin (see FIG. 4). In this embodiment, the microprojections 34 are formed by etching or punching a plurality of microprojections 34 from a thin metal sheet 36 and bending the microprojections 34 out of the plane of the sheet 36.

In one embodiment of the invention, the microprojection member 30 has a microprojection density of at least approximately 10 microprojections/cm², more preferably, in the range of at least approximately 200-3000 microprojections/cm². Preferably, the number of openings per unit area through which the agent passes is at least approximately 10 openings/cm² and less than about 3000 openings/cm².

As indicated, the microprojections 34 preferably have a projection length less than 1000 microns. In one embodiment, the microprojections 34 have a projection length of less than 500 microns, more preferably, less than 250 microns.

In a further embodiment adapted to minimize bleeding and irritation, the microprojections preferably have a projection length less than 145 microns, more preferably, in the range of approximately 50-145 microns, and even more preferably, in the range of approximately 70-140 microns.

The microprojections 34 also preferably have a width, designated “W” in FIG. 2, in the range of approximately 25-500 microns and thickness in the range of approximately 10-100 microns.

Referring now to FIG. 5, there is shown another embodiment of a microprojection member 50 that can be employed within the scope of the invention. The microprojection member 50 similarly includes a microprojection array 52 having a plurality of microprojections 54. The microprojections 54 preferably extend at substantially a 90° angle from the sheet 51, which similarly includes openings 56.

As illustrated in FIG. 5, several of the microprojections 54 include a retention member or anchor 58 disposed proximate the leading edge. As indicated above, the retention member 58 facilitates adherence of the microprojection member 50 to the subject's skin.

The microprojection members (e.g., 30, 50) can be manufactured from various metals, such as stainless steel, titanium, nickel titanium alloys, or similar biocompatible materials, such as polymeric materials. Preferably, the microprojection member is manufactured out of titanium.

According to the invention, the microprojection members can also be constructed out of a non-conductive material, such as a polymer. Alternatively, the microprojection member can be coated with a non-conductive material, such as Parylene®, or a hydrophobic material, such as Teflon®, silicon or other low energy material. The noted hydrophobic materials and associated base (e.g., photoreist) layers are set forth in U.S. application Ser. No. 60/484,142, which is incorporated by reference herein.

Microprojection members that can be employed with the present invention include, but are not limited to, the members disclosed in U.S. Pat. Nos. 6,083,196, 6,050,988 and 6,091,975, and U.S. Pat. Pub. No. 2002/0016562, which are incorporated by reference herein in their entirety.

Other microprojection members that can be employed with the present invention include members formed by etching silicon using silicon chip etching techniques or by molding plastic using etched micro-molds, such as the members disclosed U.S. Pat. No. 5,879,326, which is incorporated by reference herein in its entirety.

Referring now to FIG. 3, there is shown the microprojection member 30 having microprojections 34 coated with a biocompatible coating 35. According to the invention, the coating 35 can partially or completely cover each microprojection 34. For example, the coating 35 can be in a dry pattern coating on the microprojections 34. The coating 35 can also be applied before or after the microprojections 34 are formed.

According to the invention, the coating 35 can be applied to the microprojections 34 by a variety of known methods. Preferably, the coating is only applied to those portions the microprojection member 30 or microprojections 34 that pierce the skin (e.g., tips 39).

One such coating method comprises dip-coating. Dip-coating can be described as a means to coat the microprojections by partially or totally immersing the microprojections 34 into a coating solution. By use of a partial immersion technique, it is possible to limit the coating 35 to only the tips 39 of the microprojections 34.

A further coating method comprises roller coating, which employs a roller coating mechanism that similarly limits the coating 35 to the tips 39 of the microprojections 34. The roller coating method is disclosed in U.S. application Ser. No. 10/099,604 (Pub. No. 2002/0132054), which is incorporated by reference herein in its entirety. As discussed in detail in the noted application, the disclosed roller coating method provides a smooth coating that is not easily dislodged from the microprojections 34 during skin piercing.

According to the invention, the microprojections 34 can further include means adapted to receive and/or enhance the volume of the coating 35, such as apertures (not shown), grooves (not shown), surface irregularities (not shown) or similar modifications, wherein the means provides increased surface area upon which a greater amount of coating can be deposited.

A further coating method that can be employed within the scope of the present invention comprises spray coating. According to the invention, spray coating can encompass formation of an aerosol suspension of the coating composition. In one embodiment, an aerosol suspension having a droplet size of about 10 to 200 picoliters is sprayed onto the microprojections 10 and then dried.

Pattern coating can also be employed to coat the microprojections 34. The pattern coating can be applied using a dispensing system for positioning the deposited liquid onto the microprojection surface. The quantity of the deposited liquid is preferably in the range of 0.1 to 20 nanoliters/microprojection. Examples of suitable precision-metered liquid dispensers are disclosed in U.S. Pat. Nos. 5,916,524; 5,743,960; 5,741,554; and 5,738,728; which are fully incorporated by reference herein.

Microprojection coating formulations or solutions can also be applied using ink jet technology using known solenoid valve dispensers, optional fluid motive means and positioning means which is generally controlled by use of an electric field. Other liquid dispensing technology from the printing industry or similar liquid dispensing technology known in the art can be used for applying the pattern coating of this invention.

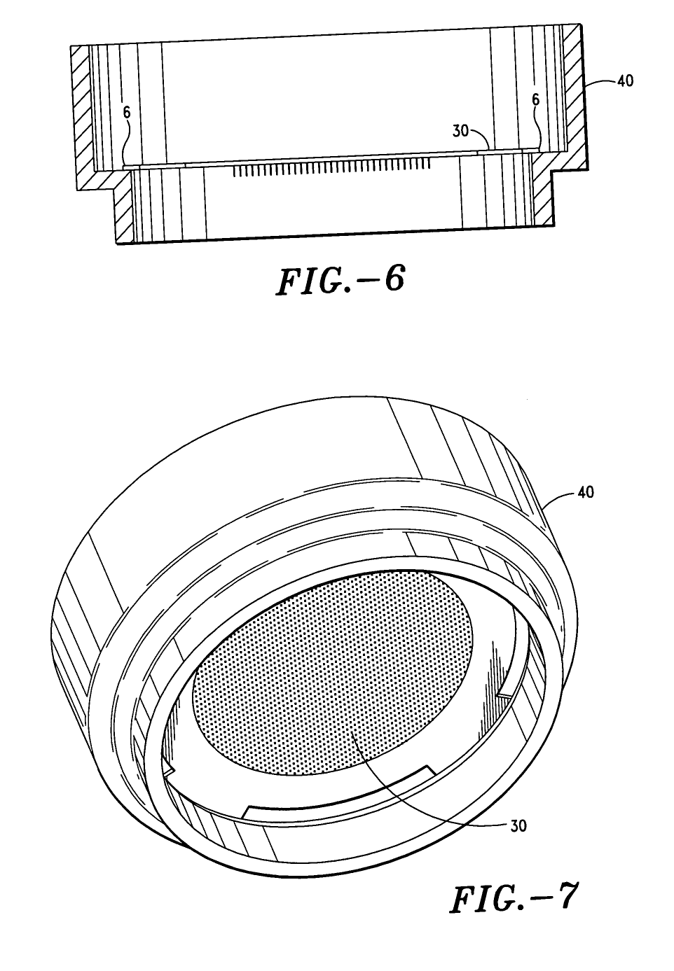

Referring now to FIGS. 6 and 7, for storage and application, the microprojection member 30 is preferably suspended in a retainer ring 40 by adhesive tabs 6, as described in detail in Co-Pending U.S. application Ser. No. 09/976,762 (Pub. No. 2002/0091357), which is incorporated by reference herein in its entirety.

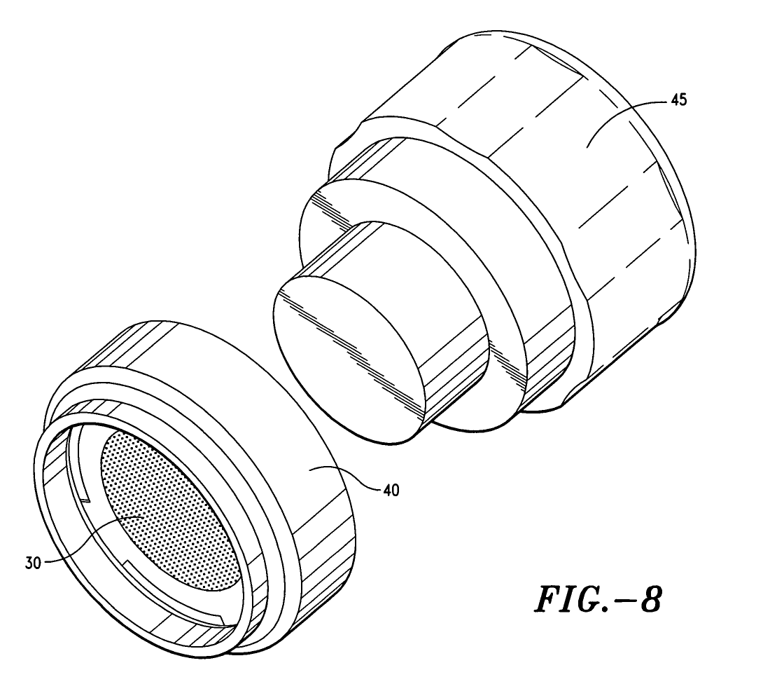

After placement of the microprojection member 30 in the retainer ring 40, the microprojection member 30 is applied to the patient's skin. Preferably, the microprojection member 30 is applied to the skin using an impact applicator 45, such as shown in FIG. 8 and disclosed in Co-Pending U.S. application Ser. No. 09/976,798, which is incorporated by reference herein in its entirety.

As indicated, in a preferred embodiment of the invention, the coating formulation applied to the microprojection member 30 to form a solid coating comprises an aqueous formulation. In an alternative embodiment, the coating formulation comprises a non-aqueous formulation. According to the invention, the immunologically active agent can be dissolved within a biocompatible carrier or suspended within the carrier.

As indicated, in a preferred embodiment of the invention, the immunologically active agent comprises an influenza vaccine. More preferably, a trivalent influenza vaccine.

In alternative embodiments of the invention, the immunologically active agent comprises a vaccine (or antigenic agent) selected from the group consisting of viruses and bacteria, protein-based vaccines, polysaccharide-based vaccine, and nucleic acid-based vaccines.

Suitable antigenic agents include, without limitation, antigens in the form of proteins, polysaccharide conjugates, oligosaccharides, and lipoproteins. These subunit vaccines in include Bordetella pertussis (recombinant PT accince—acellular), Clostridium tetani (purified, recombinant), Corynebacterium diphtheriae (purified, recombinant), Cytomegalovirus (glycoprotein subunit), Group A streptococcus (glycoprotein subunit, glycoconjugate Group A polysaccharide with tetanus toxoid, M protein/peptides linked to toxing subunit carriers, M protein, multivalent type-specific epitopes, cysteine protease, C5a peptidase), Hepatitis B virus (recombinant Pre SI, Pre-S2, S, recombinant core protein), Hepatitis C virus (recombinant—expressed surface proteins and epitopes), Human papillomavirus (Capsid protein, TA-GN recombinant protein L2 and E7 [from HPV-6], MEDI-501 recombinant VLP L1 from HPV-11, Quadrivalent recombinant BLP L1 [from HPV-6], HPV-11, HPV-16, and HPV-18, LAMP-E7 [from HPV-16]), Legionella pneumophila (purified bacterial surface protein), Neisseria meningitides (glycoconjugate with tetanus toxoid), Pseudomonas aeruginosa (synthetic peptides), Rubella virus (synthetic peptide), Streptococcus pneumoniae (glyconconjugate [1, 4, 5, 6B, 9N, 14, 18C, 19V, 23F] conjugated to meningococcal B OMP, glycoconjugate [4, 6B, 9V, 14, 18C, 19F, 23F] conjugated to CRM197, glycoconjugate [1, 4, 5, 6B, 9V, 14, 18C, 19F, 23F] conjugated to CRM1970, Treponema pallidum (surface lipoproteins), Varicella zoster virus (subunit, glycoproteins), and Vibrio cholerae (conjugate lipopolysaccharide).

Whole virus or bacteria include, without limitation, weakened or killed viruses, such as cytomegalo virus, hepatitis B virus, hepatitis C virus, human papillomavirus, rubella virus, and varicella zoster, weakened or killed bacteria, such as bordetella pertussis, clostridium tetani, corynebacterium diphtheriae, group A streptococcus, legionella pneumophila, neisseria meningitis, pseudomonas aeruginosa, streptococcus pneumoniae, treponema pallidum, and vibrio cholerae, and mixtures thereof.

Additional commercially available vaccines, which contain antigenic agents, include, without limitation, flu vaccines, Lyme disease vaccine, rabies vaccine, measles vaccine, mumps vaccine, chicken pox vaccine, small pox vaccine, hepatitis vaccine, pertussis vaccine, and diphtheria vaccine.

Vaccines comprising nucleic acids include, without limitation, single-stranded and double-stranded nucleic acids, such as, for example, supercoiled plasmid DNA; linear plasmid DNA; cosmids; bacterial artificial chromosomes (BACs); yeast artificial chromosomes (YACs); mammalian artificial chromosomes; and RNA molecules, such as, for example, mRNA. The size of the nucleic acid can be up to thousands of kilobases. In addition, in certain embodiments of the invention, the nucleic acid can be coupled with a proteinaceous agent or can include one or more chemical modifications, such as, for example, phosphorothioate moieties. The encoding sequence of the nucleic acid comprises the sequence of the antigen against which the immune response is desired. In addition, in the case of DNA, promoter and polyadenylation sequences are also incorporated in the vaccine construct. The antigen that can be encoded include all antigenic components of infectious diseases, pathogens, as well as cancer antigens. The nucleic acids thus find application, for example, in the fields of infectious diseases, cancers, allergies, autoimmune, and inflammatory diseases.

Suitable immune response augmenting adjuvants which, together with the vaccine antigen, can comprise the vaccine include, without limitation, aluminum phosphate gel; aluminum hydroxide; algal glucan: β-glucan; cholera toxin B subunit; CRL1005: ABA block polymer with mean values of x=8 and y=205; gamma inulin: linear (unbranched) β-D(2->1) polyfructofuranoxyl-α-D-glucose; Gerbu adjuvant: N-acetylglucosamine-(β 14)-N-acetylmuramyl-L-alanyl-D-glutamine (GMDP), dimethyl dioctadecylammonium chloride (DDA), zinc L-proline salt complex (Zn-Pro-8); Imiquimod (1-(2-methypropyl)-1H-imidazo[4,5-c]quinolin-4-amine; ImmTher™: N-acetylglucoaminyl-N-acetylmuramyl-L-Ala-D-isoGlu-L-Ala-glycerol dipalmitate; MTP-PE liposomes: C₅₉H₁₀₈N₆O₁₉PNa-3H₂O (MTP); Murametide: Nac-Mur-L-Ala-D-Gln-OCH₃; Pleuran: β-glucan; QS-21; S-28463: 4-amino-a, a-dimethyl-1H-imidazo[4,5-c]quinoline-1-ethanol; salvo peptide: VQGEESNDK•HCl (IL-1β 163-171 peptide); and threonyl-MDP (Termurtide™): N-acetyl muramyl-L-threonyl-D-isoglutamine, and interleukine 18, IL-2 IL-12, IL-15, Adjuvants also include DNA oligonucleotides, such as, for example, CpG containing oligonucleotides. In addition, nucleic acid sequences encoding for immuno-regulatory lymphokines such as IL-18, IL-2 IL-12, IL-15, IL-4, IL10, gamma interferon, and NF kappa B regulatory signaling proteins can be used.

According to the invention, the coating formulation can include at least one wetting agent. Suitable wetting agents include surfactants and polymers that present amphiphilic properties.

Thus, in one embodiment of the invention, the coating formulation includes at least one surfactant. According to the invention, the surfactant(s) can be zwitterionic, amphoteric, cationic, anionic, or nonionic. Examples of suitable surfactants include, sodium lauroamphoacetate, sodium dodecyl sulfate (SDS), cetylpyridinium chloride (CPC), dodecyltrimethyl ammonium chloride (TMAC), benzalkonium, chloride, polysorbates such as Tween 20 and Tween 80, other sorbitan derivatives such as sorbitan laurate, and alkoxylated alcohols such as laureth-4. Most preferred surfactants include Tween 20, Tween 80, and SDS.

In a further embodiment of the invention, the coating formulation includes at least one polymeric material or polymer that has amphiphilic properties. Examples of the noted polymers include, without limitation, cellulose derivatives, such as hydroxyethylcellulose (HEC), hydroxypropylmethylcellulose (HPMC), hydroxyl-propylcellulose (HPC), methylcellulose (MC), hydroxyethylmethylcellulose (HEMC), or ethylhydroxyethylcellulose (EHEC), as well as pluronics.

In one embodiment of the invention, the concentration of the polymer presenting amphiphilic properties is preferably in the range of approximately 0.01-20 wt. %, more preferably, in the range of approximately 0.03-10 wt. % of the coating formulation. Even more preferably, the concentration of the wetting agent is in the range of approximately 0.1-5 wt. % of the coating formulation.

As will be appreciated by one having ordinary skill in the art, the noted wetting agents can be used separately or in combinations.

According to the invention, the coating formulation can further include a hydrophilic polymer. Preferably the hydrophilic polymer is selected from the following group: dextrans, hydroxyethyl starch (HES), poly(vinyl alcohol), poly(ethylene oxide), poly(2-hydroxyethylmethacrylate), poly(n-vinyl pyrolidone), polyethylene glycol and mixtures thereof, and like polymers. As is well known in the art, the noted polymers increase viscosity.

The concentration of the hydrophilic polymer in the coating formulation is preferably in the range of approximately 0.01-50 wt. %, more preferably, in the range of approximately 0.03-30 wt. % of the coating formulation. Even more preferably, the concentration of the wetting agent is in the range of approximately 0.1-20 wt. % of the coating formulation.

According to the invention, the coating formulation can further include a biocompatible carrier such as those disclosed in Co-Pending U.S. application Ser. No. 10/127,108, which is incorporated by reference herein in its entirety. Examples of biocompatible carriers include human albumin, bioengineered human albumin, polyglutamic acid, polyaspartic acid, polyhistidine, pentosan polysulfate, polyamino acids, sucrose, trehalose, melezitose, raffinose and stachyose.

The concentration of the biocompatible carrier in the coating formulation is preferably in the range of approximately 2-70 wt. %, more preferably, in the range of approximately 5-50 wt. % of the coating formulation. Even more preferably, the concentration of the wetting agent is in the range of approximately 10-40 wt. % of the coating formulation.

The coating formulation can further include a vasoconstrictor, such as those disclosed in Co-Pending U.S. application Ser. No. 10/674,626, which is incorporated by reference herein in their entirety. As set forth in the noted Co-Pending Application, the vasoconstrictor is used to control bleeding during and after application on the microprojection member. Preferred vasoconstrictors include, but are not limited to, amidephrine, cafaminol, cyclopentamine, deoxyepinephrine, epinephrine, felypressin, indanazoline, metizoline, midodrine, naphazoline, nordefrin, octodrine, ornipressin, oxymethazoline, phenylephrine, phenylethanolamine, phenylpropanolamine, propylhexedrine, pseudoephedrine, tetrahydrozoline, tramazoline, tuaminoheptane, tymazoline, vasopressin, xylometazoline and the mixtures thereof. The most preferred vasoconstrictors include epinephrine, naphazoline, tetrahydrozoline indanazoline, metizoline, tramazoline, tymazoline, oxymetazoline and xylometazoline.

The concentration of the vasoconstrictor, if employed, is preferably in the range of approximately 0.1 wt. % to 10 wt. % of the coating.

In yet another embodiment of the invention, the coating formulation includes at least one “pathway patency modulator”, such as those disclosed in Co-Pending U.S. application Ser. No. 09/950,436, which is incorporated by reference herein in its entirety. As set forth in the noted Co-Pending Application, the pathway patency modulators prevent or diminish the skin's natural healing processes thereby preventing the closure of the pathways or microslits formed in the stratum corneum by the microprojection member array. Examples of pathway patency modulators include, without limitation, osmotic agents (e.g., sodium chloride), and zwitterionic compounds (e.g., amino acids).

The term “pathway patency modulator”, as defined in the Co-Pending Application, further includes anti-inflammatory agents, such as betamethasone 21-phosphate disodium salt, triamcinolone acetonide 21-disodium phosphate, hydrocortamate hydrochloride, hydrocortisone 21-phosphate disodium salt, methylprednisolone 21-phosphate disodium salt, methylprednisolone 21-succinaate sodium salt, paramethasone disodium phosphate and prednisolone 21-succinate sodium salt, and anticoagulants, such as citric acid, citrate salts (e.g., sodium citrate), dextrin sulfate sodium, aspirin and EDTA.

According to the invention, the coating formulation can also include a non-aqueous solvent, such as ethanol, chloroform, ether, propylene glycol, polyethylene glycol and the like, dyes, pigments, inert fillers, permeation enhancers, excipients, and other conventional components of pharmaceutical products or transdermal devices known in the art.

Other known formulation adjuvants can also be added to the coating formulation as long as they do not adversely affect the necessary solubility and viscosity characteristics of the coating formulation and the physical integrity of the dried coating.

Preferably, the coating formulation has a viscosity less than approximately 5 in order to effectively coat each microprojection 10. More preferably, the coating formulations have a viscosity in the range of approximately 0.3-2.0 poise.

According to the invention, the coating thickness is preferably less than 100 microns, more preferably less than 50 microns. Even more preferably, the coating thickness is in the range of approximately 2-30 microns

The desired coating thickness is dependent upon several factors, including the required dosage and, hence, coating thickness necessary to deliver the dosage, the density of the microprojections per unit area of the sheet, the viscosity and concentration of the coating formulation and the coating method chosen.

In all cases, after a coating has been applied, the coating formulation can be dried on the microprojections by various means. In one embodiment of the invention, the coated microprojection member (e.g., 30) is air-dried in ambient room conditions. In another embodiment, the coated microprojection member is vacuum-dried. In yet another embodiment, the coated microprojection member is air-dried and vacuum-dried thereafter.

Various temperatures and humidity levels can also be employed to dry the coating formulation on the microprojections. The coated microprojection member 30 can thus be heated, lyophilized, freeze dried or subjected to similar techniques to remove the water from the coating.

### STUDIES/EXAMPLES

The following studies and examples illustrate the apparatus, formulations methods and processes of the invention. The examples are for illustrative purposes only and are not meant to limit the scope of the invention in any way.

Referring first to Table I, there is shown a summary of the monovalent strains (i.e., lots) that were obtained and employed in the studies set forth below:

TABLE I

| | | HA Concen- | % HA of |
|---|---|---|---|
| | | tration | total vaccine |
| Strain | Lot # | (μg/mL) | content |
| B/Yamanashi/ | 5096PD | 79 | 20 |
| Fluzone ® | | | |
| B/Victoria/ | U2603 | 197 | 25 |
| Fluzone ® | | | |
| B/Victoria/ | U02995 | 210 | 18 |
| Fluzone ® | | | |
| A/New Caledonia/ | 5095PD | 95 | 16 |
| Fluzone ® | | | |
| A/Panama/ | 5094PD | 112 | 40 |
| Fluzone ® | | | |
| A/Panama/ | U02598 | 401 | 50 |
| Fluzone ® | | | |
| A/Panama/ | FA106821 | 180 | 38 |
| Vaxigrip ™ | | | |
| A/Panama/ | FA107640 | 159(123) | (33)* |
| Vaxigrip ™ | | | |
| A/New Caledonia/ | FA106076 | 131(127) | (32)* |
| Vaxigrip ™ | | | |
| B/Shangdong/ | FA107994 | 191(260, 234) | (63)* |
| Vaxigrip ™ | | | |

Pre-Formulation Process

The first bulk vaccine obtained was a monovalent A/Panama/2007/99 strain (Fluzone®) at 400 gHA/mL. The solution was turbid as received, suggesting the presence of insoluble particles due possibly to water-insoluble lipids, lipids-protein complexes, and aggregated proteins. BCA analysis, as well as dialysis of the monovalent indicated that salts and other low molecular weigh materials took up the majority of the solids content. In order to enrich the HA content of the coating to meet the dose requirements, these low MW components had to be removed. A diafiltration/concentration process was thus developed to address this issue.

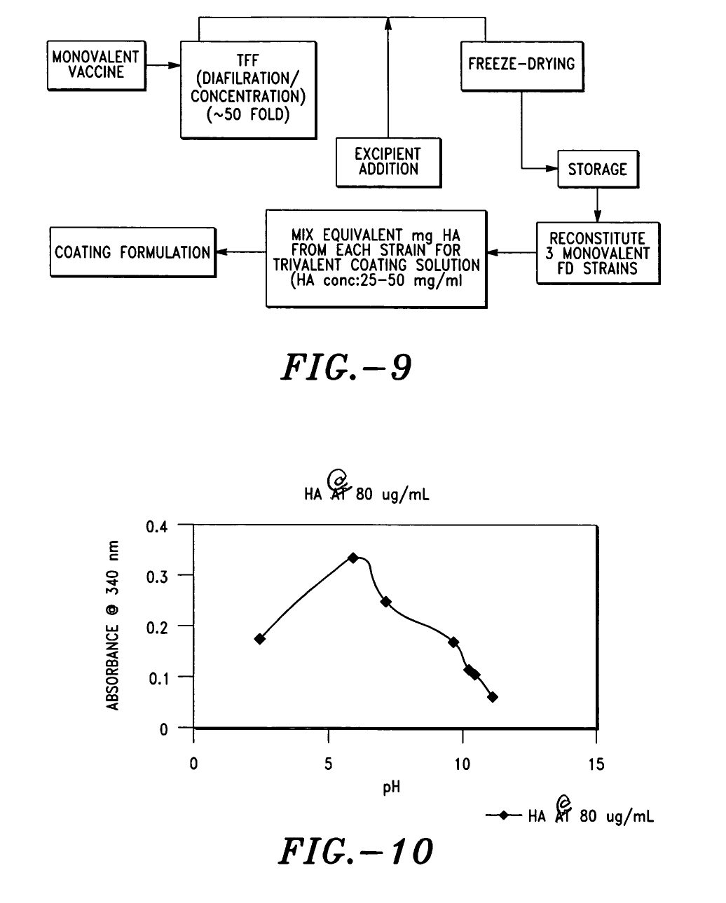

Referring now to FIG. 9, there is shown a flow diagram of the pre-formulation process that was employed. The steps set forth in the flow diagram are discussed below.

Tangential-Flow Filtration (TFF)

As is known in the art, TFF allows diafiltration and concentration to be performed at the same time. Diafiltration was used to remove low molecular weight materials. A TFF system (Millipore, Labscale) equipped with a Pellicon XL, regenerated cellulose membrane (Millipore, 50 cm², 30 kD MWCO) was set up and evaluated for the diafiltration and concentration of the vaccine raw material. The volume of the vaccine solution was reduced to 1/20^{th}- 1/50^{th} of the original volume, increasing the HA concentration to 5-10 mg HA/mL. Buffer solution was added for buffer exchange and concentration.

Lyophilization

Following tangential-flow filtration, the concentrated vaccine was formulated with lyoprotective excipients, such as sucrose or trehalose, filled into 20 mL glass vials, flash frozen with liquid nitrogen and placed on a manifold-style freeze drier (Virtis, 25EL Freezemobile). The vials were allowed to freeze-dry for 2-5 days until the chamber pressure reached a steady state (˜50 mTorr).

The noted pre-formulation process provided highly concentrated and solid-state hemagglutinin (HA) formulations as intermediate products. Indeed, the concentration of the HA formulations was at least 500-fold the concentration of the commercial product. The noted intermediate products were also highly potent and immunologenic.

As will be appreciated by one have ordinary skill in the art, the noted pre-formulation process of the invention can be modified and adapted to pre-formulate various vaccine source materials and forms thereof. For example, the process could be adapted to use raw materials received at higher concentrations. In this case, the diafiltration step would not be necessary and the high concentration raw materials would be directly lyophilized and reconstituted to produce the coating formulation.

The pre-formulation process could also be adapted to use raw materials received as solids such as, but not limited to lyophilized or spray dried powders. In this case, the solid raw materials would be directly reconstituted to produce the coating solution formulation.

The pre-formulation process could also be modified for use with high purity raw materials, such as, but not limited to, cell derived influenza vaccines. In this case the materials may be of sufficient purity that the lyophilization and reconstitution step would be unnecessary.

Formulation Development

The formulation effort was directed to developing a coating formulation with suitable coating properties and stability, defining a coating system that can reliably produce reproducible coating dose, and identifying an array design that can deliver the vaccine with good delivery efficiency and acceptable skin tolerability.

Coating Process

Two types of the coater were used in the study. The first coater, was fitted with a 0.38″ diameter drum made of Delrin. The drum is submerged in a reservoir that has a loading volume of 0.25-mL. This reservoir has no chilling capability, but allows for the direct infusion of fresh water to compensate for evaporation during operation. The thickness of the film established on the drum is ˜200-250 μm.

The second coater evaluated was fitted with a 0.621″ diameter stainless steel drum and a concentric reservoir. The reservoir for this coater has a loading volume of 0.3-0.7 mL, depending on the diameter of the drum. The drum diameter also controls the thickness of the film, which is ˜80-90 μ for the 0.621″ drum. The reservoir of this coater is equipped with thermo-electrical chilling (TEC). By controlling the drum temperature at the dew point of the ambient condition, the changes in the concentration of the coating solution can be minimized. Coating height was determined by the sum of microprojection length and array strip thickness.

Microprojection Array Designs

Eight microprojections arrays were employed in the formulation development. The microprojection array designs varied in microprojection length, tip angle, and the presence of additional design features, such as retention barbs, and/or microprojection stops. Two microprojection array designs, MF-1 and MF-2, were initially evaluated.

Excipients

To evaluate whether the microprojections could be coated using a suspension, i.e., non-clear coating solution, the initial focus was on stabilizing the in-soluble particles by adding a surfactant.

Referring now to Table II, there is shown the effects of surfactants in reducing solution turbidity. The noted data suggests that adding a surfactant could help particle disaggregation/solubilization, as determined by a reduction in solution turbidity. The order of surfactant strength is SDS>Triton X100>Tween 20, which is consistent with solution clarity in the presence of the same surfactants (see Table III).

TABLE II

| Turbidity @ | | Bulk/0.1% | Bulk/0.1% | Bulk/0.1% |
|---|---|---|---|---|
| 340 nm | Bulk | SDS | Triton X100 | Tween 20 |
| 80 μg/mL HA | 0.279 | 0.022 | 0.053 | 0.185 |

TABLE III

| | | Membrane/ | | |
|---|---|---|---|---|
| Surfactant | Appearance | MWCO | Final Conc. | Comments |
| SDS | Clear | Ultrfree/ | 30 mg/mL | Viscosity |
| | | 10 kD + | | exceeding |
| | | Centricon | | 100 centi- |
| | | 30 kD | | poise |
| Triton X | Clear | Ultrfree/ | 15 mg/mL | Gel up |
| | | 10 kD | | |
| Tween 80 | Turbid | Ultrfree/ | 15 mg/mL | Gel up |
| | | 10 kD | | |

Another potent class of surfactant, Zwittergent, is also capable of breaking protein/lipid-based aggregates. Table IV lists three types of Zwittergents whose solubilizing power increases with increasing hydrophobicity of the Zwittergent, i.e., Zwittergent 3-14 is the strongest.

TABLE IV

| Zwittergent | Absorbance @ 340 nm |
|---|---|
| Starting vaccine material | 0.3557 |
| (0.2 mg/mL HA) | |
| 3-10 | 0.120 |
| 3-12 | 0.087 |
| 3-14 | 0.070 |

Adjusting the pH was also shown to decrease the vaccine's turbidity at high and low pH, as shown in FIG. 10. However, a large increase or decrease in pH could compromise the stability of the antigen at high concentration. Therefore, a significant deviation from pH 7.2 in order to remove the solution turbidity was not employed.

With the pre-formulation process permitting the vaccine to be concentrated to the required level for coating, along with the strategy of preparing solubilized or suspended coating solutions, seven candidate formulations were further investigated. The formulations, which are set forth in Table V, contain at least one or more excipients.

TABLE V

| Ref No. | Formulation (Form) |
|---|---|
| 1 | 5% HA/1% trehalose/10% SDS |
| | (solubilized) |
| 2 | 5% HA/1% trehalose/10% Triton X100 |
| | (solubilized) |
| 3 | 5% HA/1% trehalose/5% Zwittergent |
| | 3-14/pH10 (Na₂CO₃-NaHCO₃) |
| | (solubilized) |
| 4 | 5% HA/1% trehalose/10% Zwittergent |
| | 3-14 (solubilized) |
| 5 | 5% HA/5% sucrose/2% Tween 80 |
| | (suspension) |
| 6 | 5% HA/5% sucrose (suspension) |
| 7 | 5% HA/2.5 trehalose/2.5 mannitol/2% |
| | Pluronic F68 (suspension) |

Formulations 1-4 were solubilized solutions. Formulations 5-7 were suspension/turpid solutions. All formulations contained at least a sugar to stabilize the protein. Formulation contained a weak surfactant, Tween 80, which, it was believed, could provide increased solubilization of the vaccine and perhaps increased immunogenicity. Formulation 6, containing only sucrose, was the simplest formulation of all the formulations evaluated. Formulation 7 included mannitol and a solid surfactant, Pluronic F68, which, it was believed could decrease the hygroscopicity of the coating and increase the coating integrity/physical stability.

Coating Solution/Suspension Characterization

As is well recognized in the art, two physical parameters primarily govern coating feasibility and wettability of the coating solution. Each of the noted parameter is discussed below.

Viscosity

Solution viscosity affects the flow of the coating solution during microprojection coating. If the coating solution viscosity is too low, a significant portion of the liquid may drip back into the reservoir when the submerged microprojection array is removed from the coating solution before the liquid has a chance to form a film around the tip of the microprojections. This will result in less efficient process requiring many more cycles of coating.

On the other hand, if the coating solution viscosity is too high, the liquid on the microprojection array will move very slowly and may result in odd coating morphology. Table VI summarizes the composition of the seven candidate formulations in the solid state. All seven coating solution formulations contained 2-phenoxylethanol at 6 mg/mL as a preservative. The HA content in the coating solution were ˜30% in this case where HA purity is 50%.

TABLE VI

| | Formulation | | | | | | |
|---|---|---|---|---|---|---|---|
| | 1 | 2 | 3 | 4 | 5 | 6 | 7 |
| HA | 23.1 | 23.1 | 30.1 | 23.1 | 28.4 | 32.1 | 28.4 |
| Sugar(s) | 4.6 | 4.6 | 6.1 | 4.6 | 28.4 | 32.1 | 28.4 |
| Non-HA | 23.1 | 23.1 | 30.1 | 23.1 | 28.4 | 32.1 | 28.4 |
| materials | | | | | | | |
| Surfactant | 46.3 | 46.3 | 30.1 | 46.3 | 11.4 | 0 | 11.4 |
| 2-PE | 2.9 | 2.9 | 3.6 | 2.9 | 3.4 | 3.7 | 3.4 |

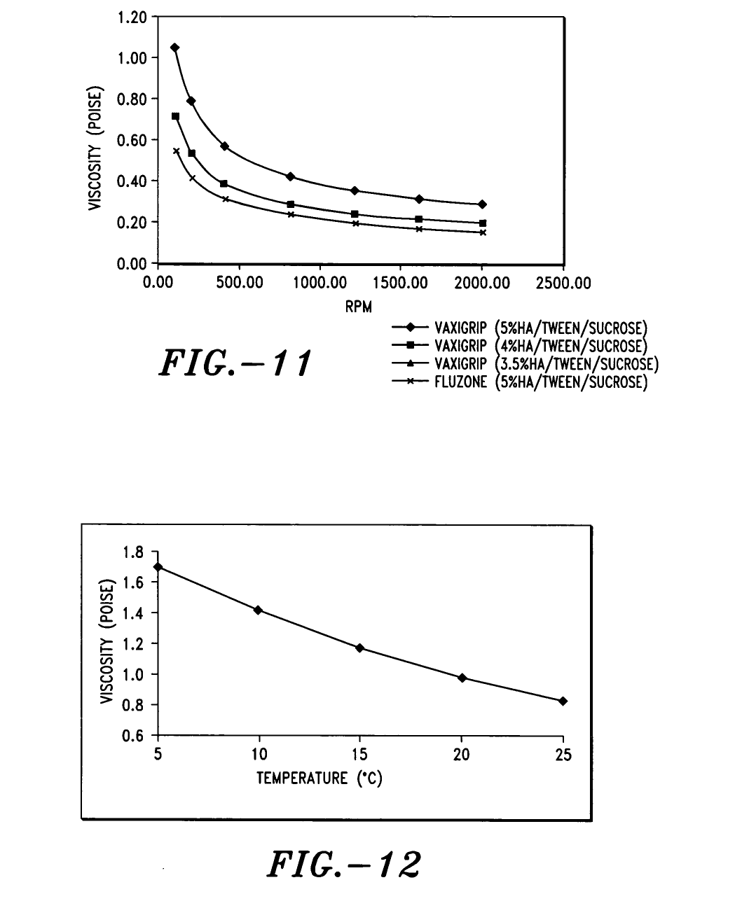

Referring now to FIG. 11, there is shown a graph comparing two different lots of vaccine; Fluzone® and Vaxigrip™. Both lots comprises A/Panama strain and were formulated into Formulation No. 5 (HA: trehalose:Tween 80=5:5:2 weight ratio).

The coating formulation was normally at 50 mg/mL (5%) of HA. However, at this concentration, the solution viscosity for the Vaxigrip™ was much higher, i.e., ˜0.8 poise at 200 rpm.

As illustrated in FIG. 11, the viscosity of the formulations decreases with dilution. At 35 mg/mL HA (3.5%), the solution viscosity of the Vaxigrip™ formulation reached the same level as the Fluzone® formulation at 50 mg/mL HA (5%), which was measured at ˜0.4 poise at 200 rpm.

Other than HA purity and HA concentration, the temperature of the coating solution is another important factor affecting viscosity. A highly viscous coating solution comprising an A/New Caledonia strain having 15% HA purity was thus prepared by reconstituting the freeze-dried vaccine to 22.5 mg/mL of HA (a modified Formulation No. 6 with 2.25% HA/2.25 sucrose). The viscosity of this coating solution was measured at several temperatures below room temperature (see FIG. 12). The solution was highly viscous, i.e., ˜1.70 cp at 5° C.

As is well known in the art, temperature is an important parameter in the coating system as the stainless steel solution reservoir and the drum are temperature controlled at the dew point of the ambient environment for the purpose of minimizing water loss due to evaporation during the coating process. The dew point under normal ambient conditions (22° C. and 30-45% RH) is typically in the range of 4-10° C.

Although solution viscosity may vary significantly, it has been found that the coating solution can be readily and efficiently coated on a microprojection array over a wide range of viscosity, preferably in the range of approximately 0.3-2.0 poise.

Wettability: Contact Angle

As is known in the art, wettability determines the ability of the liquid to attach, adhere, and spread over the surface to be coated. Contact angle measurements of liquid droplets on substrate surfaces are commonly used to characterize surface wettability. The measured contact angles are referenced to pure water whose contact angle under the same condition is ˜70-80°. Generally, the smaller the contact angle, the better the wettability.

Referring now to Table VII, there is shown the contact angles of the seven influenza vaccine formulations identified in Table V on a metallic titanium surface, which had not been cleaned. Compared to pure water, all formulations showed good wettability with contact angles ranging from 26° to 36°. This narrow range of contact angles of very different formulation suggests that contributions of the vaccine to the wettability might outplay contribution from the excipients. To verify this hypothesis, the contact angles of the same formulations in the absence of the vaccine were measured. The results suggest that components in the vaccine appear to help wet the metal surface. Without the vaccine, these excipients, except for the potent surfactants, were not able to wet the metal surface effectively.

TABLE VII

| | Contact | Contact angle |
|---|---|---|
| | angle | Without the vaccine |
| Formulation | (°) | (°) |
| Water | 72 | 72 |
| (1) 5% HA/10% SDS | 32 | ND |
| (2) 5% HA/10% Triton X100/pH 10 | 36 | ND |
| (3) 5% HA/5% Zwittergent 3-14/pH 10 | 28 | 30 |
| (4) 5% HA/5% Zwittergent 3-14 | 26 | ND |
| (5) 5% HA/5% sucrose/2% Tween 80 | 31 | 40 |
| (6) 5% HA/5% sucrose | 34 | 60 |
| (7) 5% HA/2.5% trehalose/2.5% mannitol/ | 32 | 59 |
| 2% pluronic F68 | | |

Overall, the coating solution exhibited robust wetting properties, which were minimally affected by the coated surface, and showed excellent coating properties despite the contact angle being at the low end of the optimum contact angle range. The optimum contact angle was deemed to be in the range of approximately 30-60°, which was established from other biopharmaceutical and placebo formulations.

Candidate Selection for Immunogenicity Studies

The selection of final formulations for immunogenicity studies was based on antigen stability and delivery performance.

Trivalent Formulation

Referring back to Table 1, HA purity of each lot was determined. The HA purity ranged from 16% to 50%. Based on recognized empirical relationships, HA content of the coating solution decreases dramatically from ˜30% to 11% if the HA purity decreases from the desired 50% to 20%. Despite such HA purity variations, these materials could all be successfully processed, suggesting the robustness of the pre-formulation process.

Two approaches were evaluated for the preparation of the trivalent flu vaccine from three monovalent strains, A/Panama/Fluzone®, A/New Caledonia/Fluzone® and B/Victoria/Fluzone®. In the first approach, the three monovalent strain starting materials, A/Panama/Fluzone®, A/New Caledonia/Fluzone® and B/victoria/Fluzone®, were processed separately to provide three freeze-dried monovalent intermediates. Freeze-dried material from each of the three intermediates, of equivalent HA amount, were combined and reconstituted with water for coating.

The second approach was performed by mixing the three monovalent starting materials of equivalent HA amount, i.e., different volumes. The trivalent mixture was then diafiltered and concentrated by the TTF system and freeze dried. The coating solution from the second approach had the same coating properties as that from the first approach.

Coating of the trivalent formulation (24 mg/ml HA, i.e. ˜8 mg/ml per HA strain) showed the tip-coating morphology at a similar location regardless of the microprojection array design used. Measured from the tip of the microprojections, the coating extended ˜90 μm downward for all designs, suggesting that a well-controlled coating system was established.

Characterization of Coated Microprojection Array

Other than morphology, several physical and biochemical aspects of the coating needed to be characterized to understand the performance of the formulation process. The physical parameters include water evaporation and moisture content during and after coating and microbiological considerations of the coating.

Coating Feasibility/Morphology: Solubilized Formulations (Nos. 1-4)

Despite the fact that different coating formulations could lead to various coating morphologies, similar and acceptable coating location/morphology was obtained regardless of the formulation, suggesting that the presence of some vaccine components favored the coating process as reflected by the contact angle results. Coating feasibility was demonstrated with four formulations.

Coating Feasibility/Morphology: Suspension Formulations (Nos. 5-7)

Although Formulations Nos. 5-7 were highly turbid viscous solutions, the suspension was stable since no phase separation was observed after storage under refrigeration for over one month. Furthermore, there was no clear particle sedimentation after centrifugation at 7,000 rpm for 2 minutes. A uniform thin film was formed on the drum during coating with no obvious particles observed-further evidence of a fine, stable suspension.

Moisture Content

As reflected in Table VIII, it was found that the moisture content of the coating was affected by the drying and the processing environment, particularly the relative humidity of the ambient conditions. The coating solution from Formulation 5 (HA/sucrose/Tween 80) dried on the microprojection arrays or a titanium sheet substrate resulted in 1.7% moisture content only if subjected to vacuum-drying after air-drying. Without vacuum drying, the coating's moisture content was significantly higher at 6.2%, which would vary with the humidity of the ambient air.

TABLE VIII

| | | Water |
|---|---|---|
| Sample information | Drying | Content (%) |
| Coated arrays of Formulation 5 | Air-dried and then | 1.7 |
| (A/Panama of Vaxigrip ™) | vacuum dried | |
| Formulation 5 dried on titanium | Air dried overnight | 6.2 |
| sheet | | |
| Formulation 5 dried on titanium | Air-dried for 2 hours | 1.7 |
| sheet | and then vacuum | |
| | dried overnight | |

Microbiology

Microbiology analysis was performed in the low-bioburden production area, i.e., “non-sterile”, mode for the trivalent sucrose only formation without any preservatives for a GLP production batch and a GMP production batch. The results from this analysis are set forth in Table IX.

TABLE IX

| | Trivalent Coating | | Trivalent Coated | |
|---|---|---|---|---|
| | Solution | | Arrays | |
| | | Microbial | | Microbial |
| Batch | Endotoxin* | Content* | Endotoxin | Content |
| GLP | <0.05 EU/mL | <0.04 CFU/mL | <0.5 EU/array | <1 CFU/array |
| GMP | <0.04 EU/mL | <0.05 CFU/mL | <0.5 EU/array | <1 CFU/array |

*Equivalent quantity at single human dose concentrations. Trivalent coating solution is 466 times more concentrated than currently marketed vaccine solution.

As reflected in Table IX, in the absence of any preservative, both batches of coating solution contained very low levels of endotoxin and microbial content. The results thus indicate that the processes employed to derive the coating solution and the coating process itself can be operated in such a way as to not introduce additional bioburden into the product.

SDS-PAGE/Western blot

HA antigenicity in three final formulations (Formulation Nos. 3, 6, and 7) coated on microprojections was analyzed by Western Blot analysis. Compared to the starting material (Lane 2), all coated and freeze-dried formulations displayed similar band patterns. The three bands were believed to be associated with HA as monomer (˜75 kD), or trimer (˜225 kD). Therefore, based on the matched bands and band intensity (relative to starting vaccine), it was concluded the antigen HA in formulations that had been freeze-dried and coated onto microprojection arrays maintained antigenicity.

BCA vs. SRID

As is well known in the art, SRID is the only approved assay to determined HA in vitro potency, which is, in general, consistent with immunogenicity. However, it is time consuming (3 days). To monitor HA potency during the pre-formulation and coating process in a timely fashion, the BCA protein assay was performed and compared with results from the SRID assay, which would allow short-term HA stability to be evaluated.

Referring now to Table X, there is shown a summary of BCA/SRID results for the three monvalent strains after TFF concentration, freeze-drying, reconstitution into the trivalent coating liquid, and coating. The BCA results were, in general, consistent with SRID results except in the A/New Caledonia case where the freeze-dried material had a much lower SRID value than the BCA value. However, these two values were better matched in the reconstituted trivalent liquid formulation, suggesting that the earlier inconsistency was, in all likelihood, due to sample preparation or assay variation.

TABLE X

| | A/Panama | | B/Victoria | | A/New Caledonia | |
|---|---|---|---|---|---|---|
| | BCA | SRID | BCA | SRID | BCA | SRID |
| Materials | (μg/mL) | (μg/mL) | (μg/mL) | (μg/mL) | (μg/mL) | (μg/mL) |
| Starting | 130 | 110 | 150 | 140 | 100 | 100 |
| material | | | | | | |
| TFF | 71 | UD | 49 | 55 | 62 | 74 |
| concentrated | | | | | | |
| Freeze-dried | 120 | UD | 84 | 92 | 148 | 68 |
| Trivalent | 1320 | UD | 640 | 720 | 1070 | 1260 |
| reconstituted | | | | | | |
| Trivalent | | UD | | 18.3 | | 25.3 |
| coated# | | (18.0) | | (21.2) | | (30.5) |

Microprojection Array Delivery and Skin Tolerability

Sixteen separate delivery studies were performed to assess delivery efficiency and skin tolerability. Each study is summarized in Table XI.

TABLE XI

| Study | | Skin |
|---|---|---|
| No. | Formulation | assessment |
| 1 | Fluzone ® A/P (1, 2, 4) | no |
| 2 | Fluzone ® A/P (2, 3, 5) | no |
| 3 | Fluzone ® A/P (2, 5, 7) | no |
| 4 | Fluzone ® A/P (3, 5, 7) | no |
| 5 | Vaxigrip ™ A/P (3, 5, 7) | no |
| 6 | Vaxigrip ™ A/P (5) | no |
| 7 | Vaxigrip ™ A/P (5) | no |
| 8 | Trivalent (6) | no |
| 9 | Trivalent (6) | no |
| 10 | Trivalent (6) | no |
| 11 | Fluzone ® A/P (6) | yes |
| 12 | Fluzone ® A/P (6) | yes |
| 13 | Fluzone ® A/P (6) | yes |
| 14 | Fluzone ® A/P (6) | yes |
| 15 | Fluzone ® A/P (6) | no |
| 16 | Fluzone ® A/P (6) | yes |

Delivery Studies Nos. 1-7

Delivery studies Nos. 1-7 were directed to two microprojections designs, hereinafter designated MF-1 and MF-2. The results suggest that delivery by the MF-1 microprojection design is highly effective, delivering 40-90% of the coating into the skin, regardless of the formulation.

Delivery Studies Nos. 8-15

Delivery studies Nos. 8-15 focused on microprojection designs that would offer balanced delivery and skin tolerability. As bleeding is primarily caused by penetrating too deeply, directly correlating with microprojection length, the six designs that were chosen for further evaluation (MF-3, MF-4 and MF-5), each had a microprojection length of 225 μm and a density of 1316 microprojections/2cm² array.

The investigation, which comprised eight microprojection array designs, spanned seven delivery studies to evaluate their drug delivery performance. The array designs were tested by measuring the amount of fluorescein-vaccine content present in-vivo hairless guinea pig skin with increasing drug loading.

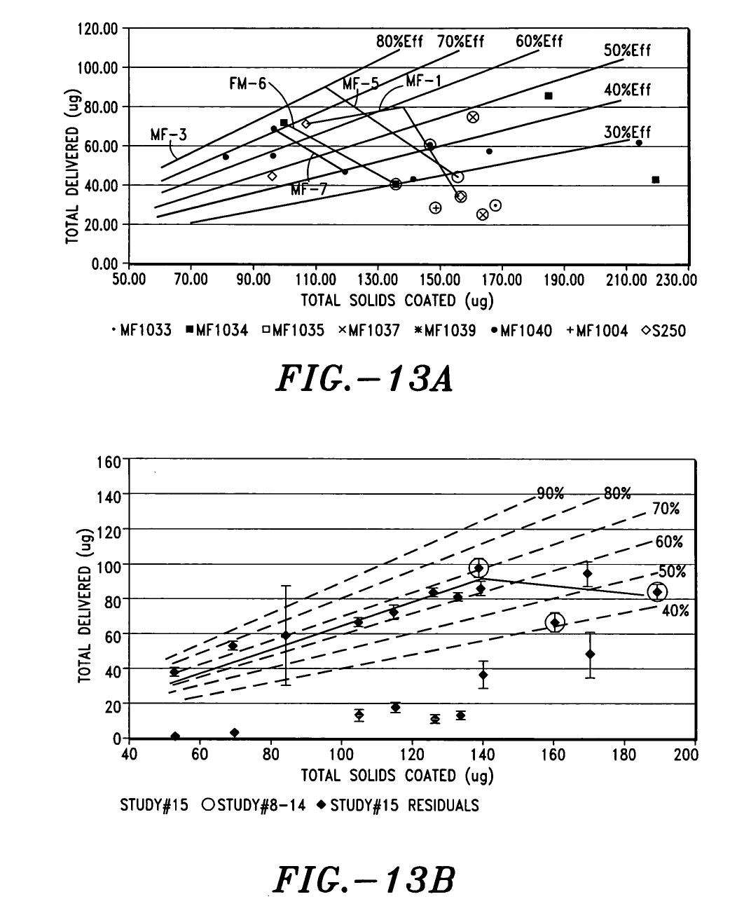

Referring now to FIG. 13A, there is shown the delivery result summary for the eight microprojection array designs. The MF-3 array design was found to maintain its high delivery efficiency, up to 140 μg of drug coating, the coating point at which the maximum amount of drugs solids can be delivered with the compared designs. The delivery efficiency of the MF-1, MF-6 and MF-7 array designs started to decrease near 100 μg of drug coating, causing the maximum amount of drug delivery with these designs to be lower than the MF-3.

To confirm the performance of MF-3, a series of MF-3 arrays was prepared for DS No. 15 with a broad range of coated amount; from 50 to 170 μg total solids coated. The delivery results shown in FIG. 13B suggest that the delivery efficiency profile for DS No. 15 almost overlaps with the efficiency profile for the MF-3 array observed in DS Nos. 8-14 (see Table XI). The delivered amounts initially follow the 70% isocline, until the inflection point at 140 μg at which point the delivered amount levels off despite an increased coating amount. Coating residues after array application were low for the smaller coated amounts, and jumped up at a coating amount of 140 μg, which is consistent with the abrupt change in coating amount delivered.

Skin tolerability (micro-bleeding) and penetration related features, such as retention, are important to assess the safety and robustness of the system. Microprojection patches were thus applied to live (duplicates for each system) and euthanized hairless guinea pigs (HGP) for 3 and 15 minutes, respectively. Upon removal of the patch, the animals were evaluated for skin reaction/micro-bleeding (live-animal only), the retention function, and penetration score at the application site dyed with methylene blue.

With regards to retention, microprojection designs with retention features (i.e., MF-3, MF4, MF-5 and MF-7) exhibited observable retention in the skin, which diminished with increasing coating amount. No bleeding was observed in any case with high coating amount (MF-3 with 160 μg of coating and MF-1 with 138 μg of coating).

The range of the coating amount was determined by antigen purity and dose to be delivered. Considering a bulk vaccine of 40% HA purity, the total coating amount including excipient would be ˜150 μg per 2 cm² array for the 45 μg HA dose and 50 μg per 2cm² array for the 15 μg HA dose.

Delivery Study No. 16

Delivery Study No. 16 was dedicated to several microprojection array designs coated with a low dose of HA, ˜15 μg/array, i.e. ˜60-70 μg of total coating per array. The study, which included four designs (MF-3, MF-5, MF-6 and MF-7), demonstrated to be most effective in high dose.

The four array designs were coated with a total coating amount of 60 - 70 μg based on the same A/Panama/sucrose formulation used in DS Nos.13 & 14. Referring now to Table XII, there is shown a comparison of the uncoated and coated arrays in terms of retention score and bleeding tendency. Retention performance was rated based on a 1-5 scoring system.

TABLE XII

| | Retention (1-5 scale) | | Bleeding | |
|---|---|---|---|---|
| Array Design | Uncoated | coated | Uncoated | coated |
| MF-6/1 | 5 | 4 | 100% | 25% |
| 2 | 5 | 5 | 100% | 50% |
| 3 | 5 | 4 | 100% | 20% |
| MF-3/1 | 4 | 3 | 75% | 25% |
| 2 | 5 | 4 | 100% | 50% |
| 3 | 4 | 4 | 75% | 25% |
| MF-5/1 | 4 | 4 | 50% | 4-5 spots |
| 2 | 3 | 4 | 25% | 0 |
| 3 | 3 | 3 | 100% | 2-3 spots |
| MF-7/1 | 1 | 3 | 2-3 spots | 0 |
| 2 | 3 | 3 | 50% | 0 |
| 3 | 3 | 1 | 100% | 0 |

The retention results suggest that (i) the uncoated arrays outperformed the coated arrays and (ii) the performance ranking followed the order of MF-6˜MF-3>MF-5>MF-5. The same trend was observed with the bleeding tendency. Overall, the MF-5 design was robust in terms of retention and penetration, and appeared to offer better skin tolerability at the low dose.

Immunogenicity Studies

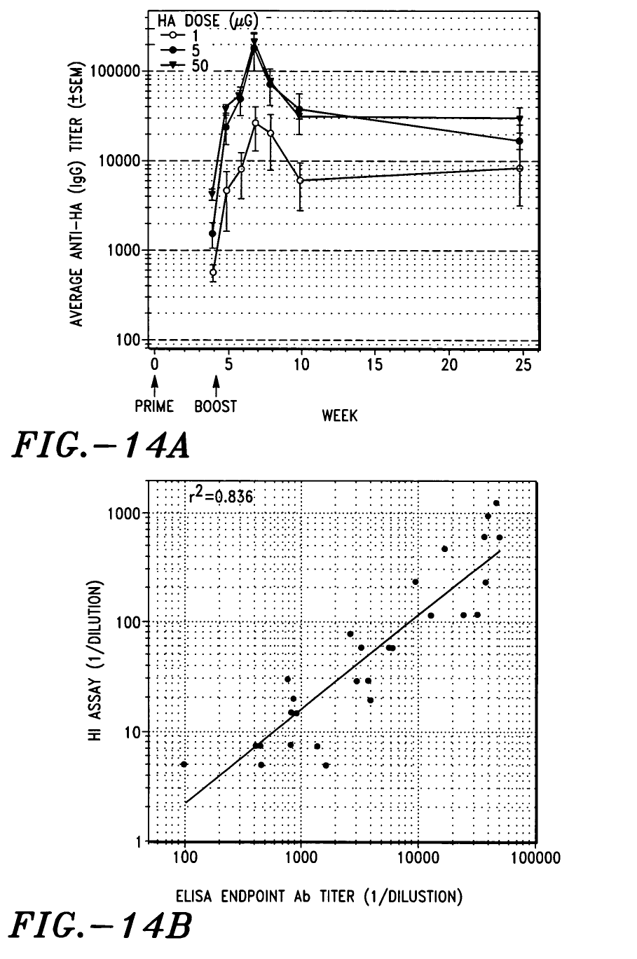

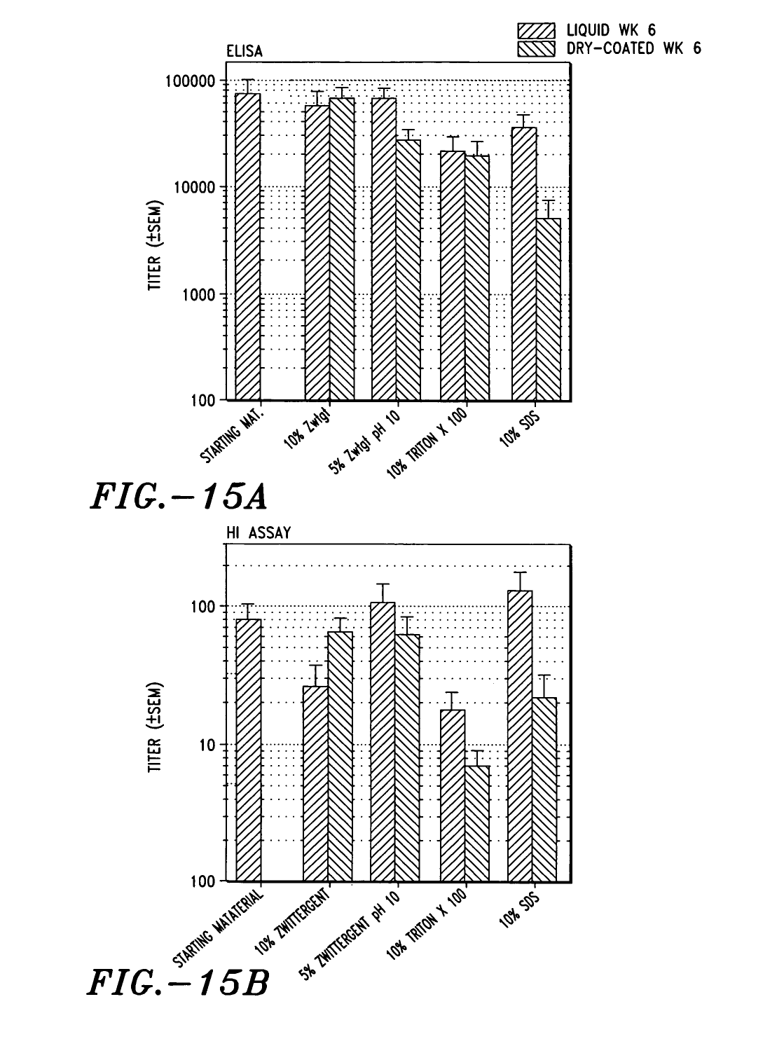

Four immunogenicity studies, in hairless guinea pigs (HGPs). The first study established the antibody response kinetics and antigen dose response using intramuscular (IM) injections at doses 1, 5 and 50 μg A/Panama (H3N2). This study demonstrated that a primary immunization with increasing HA doses from 1 to 50 μg resulted in increased antibody titers. Upon booster immunization (performed on week 4), a dose response was observed between 1 to 5 μg HA. However, no statistical difference was observed between 5 and 50 μg HA doses. Peak antibody titers were observed 2-3 weeks after the booster immunization (see FIG. 14A). A correlation was established between total HA-specific IgG titers (measured by ELISA) versus hemagglutinin inhibition (HI) activity (See FIG. 14B). Based on this data, a 5 μg HA dose was subsequently used to evaluate formulations for HA potency.

A second immunization study was conducted to evaluate the relative immunogenicity of several formulations of HA/Panama (H3N2). Four formulations containing HA/Panama (H3N2) were evaluated:
(1) 40-52 mg/mL HA, 10% zwittergent 3-14
(2) 40-52 mg/mL HA, 5% zwittergent 3-14 pH 10.
(3) 40-52 mg/mL HA, 10% Triton X100 pH 10
(4) 40-52 mg/mL HA, 10% Triton X100 pH 10

An aliquot of each concentrate was transferred onto the surface of a titanium disk and allowed to dry (i.e., “dry-coated”). Both the liquid concentrates and the dry-coated disks were tested in the study. A 0.1 mL volume (5 μg dose) of each diluted preparation was injected by IM route into HGPs on days 0 (primary) and 28 (booster). A control group was included that consisted of an equivalent 5 μg dose (starting material).

The study demonstrated that all formulations were capable of inducing anti-HA antibody responses, as measured by ELISA and HI assay (see FIGS. I SA and 1 SB). However, there were differences among the various HA formulations. Formulations containing 10% Triton X-100 (liquid or dry-coated) or 10% SDS (dry-coated) had reduced immuno-potency. All other HA preparations did not appear to statistically meaningful when compared to an equivalent injection dose using the starting material.

The third immunization study was performed to demonstrate that monovalent A/Panama (H3N2) coating formulations that were dry-coated onto microprojection arrays were capable of inducing both primary and secondary HA-specific antibody responses. IM control groups were included using the starting HA material. A single microprojection array design (MF-1 ) was used. A total of 4 HA formulations were tested at two targeted HA coatings doses on microprojection arrays (5 and 15 μg/array):
(1) HA, Zwittergent (10%), Trehalose (2.5%)
(2) HA, Tween-80 (2%), Sucrose (5%)
(3) HA, Pluronic F68 (2%), Trehalose (2.5%) Mannitol (2.5%)
(4) HA, Sucrose (5%)

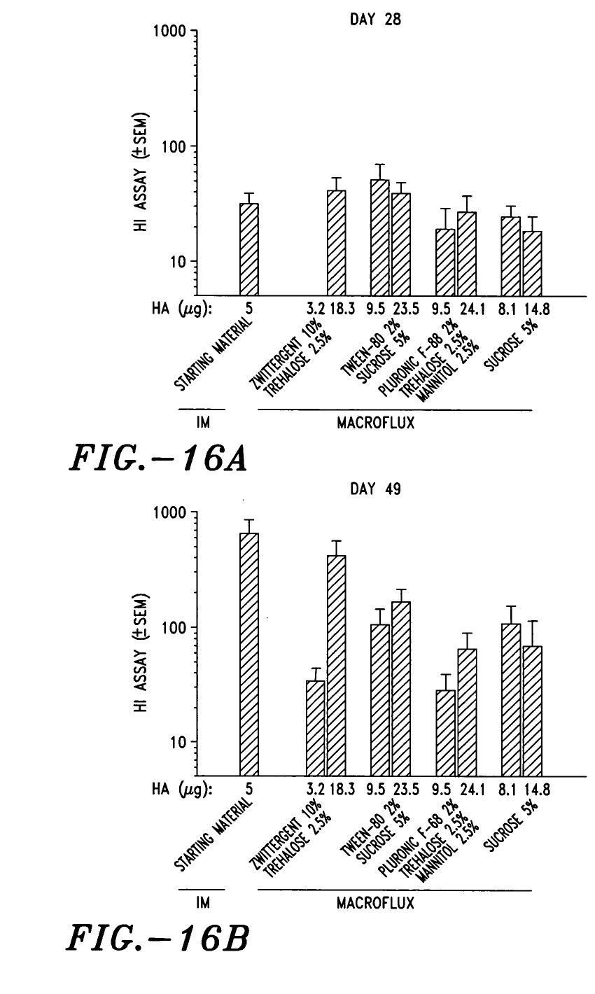

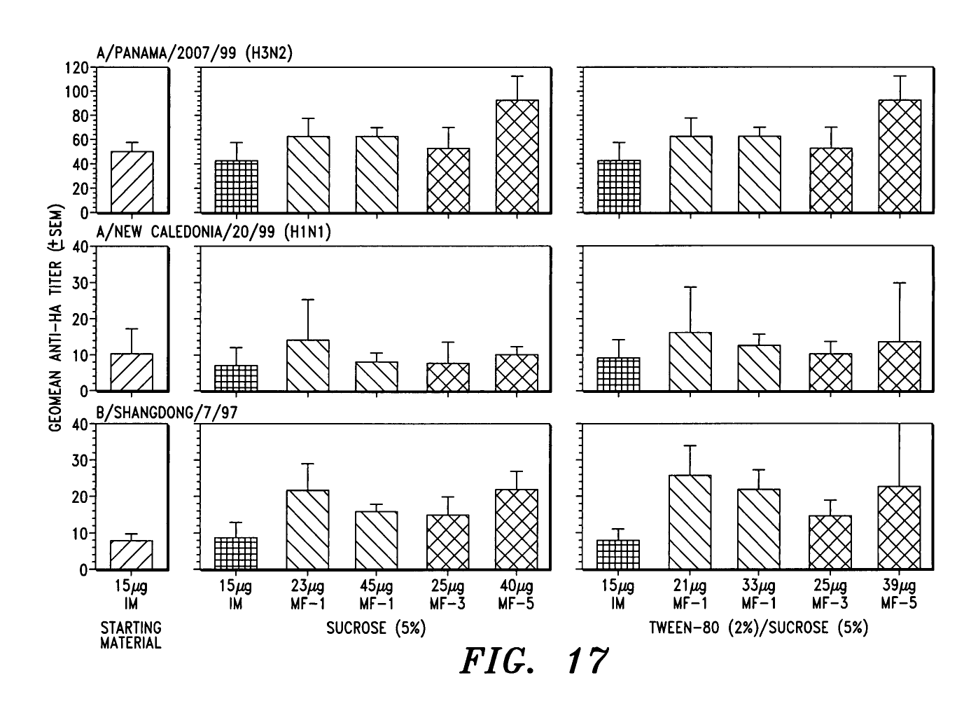

Collectively, the results (serum HAI titers) from this study demonstrated that primary (day 28) and secondary antibody (day 49) responses could be generated using HA coated Macroflux systems (see FIGS. 16A and 16B). Moreover, HA-specific serum neutralizing antibodies were generated in animals immunized with the patch. Some formulation differences could be observed at the higher targeted HA coating dose after the booster immunization. The highest mean HAI titers were generated from HGPs immunized with the HA formulation using Zwittergent 3-14/trehalose. The serum neutralizing antibody titer level from this group was most similar to the IM treatment control.

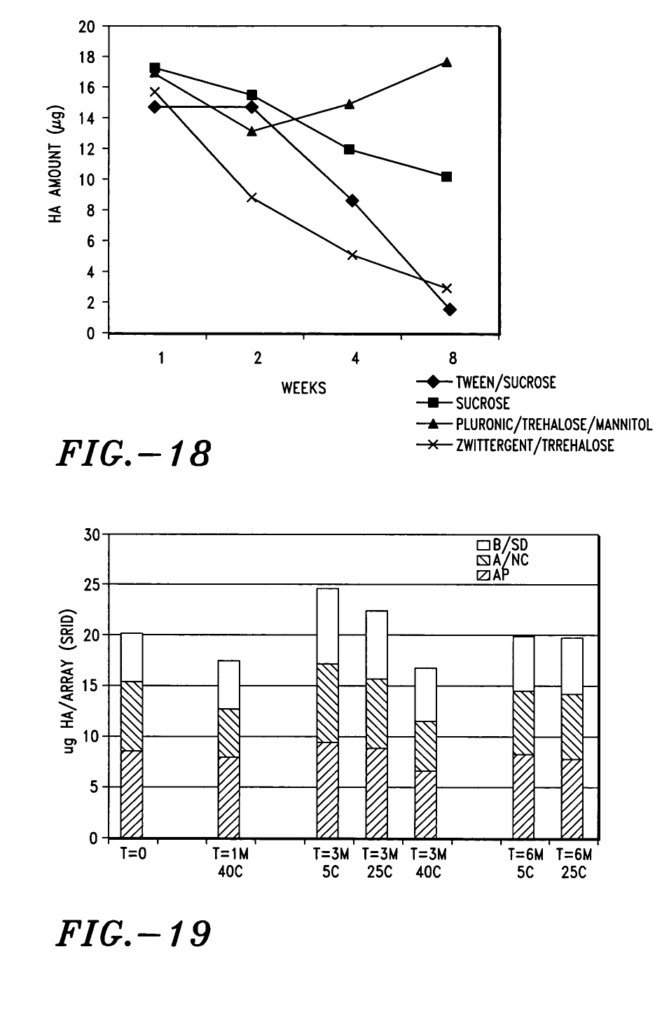

The fourth study assessed by immunogenicity testing of trivalent influenza formulations dry-coated onto titanium microprojection arrays in HGPs. The study consisted of evaluating two trivalent coating formulations, three Macroflux microprojection array designs, and two HA coating doses. The trivalent influenza formulation consisted two A strains (A/Panama/2007/99 [H3N2], and A/New Caledonia/20/99 [HIN1]), and one B strain (B/Shangdong/7/97). The HA strains were formulated at a ratio of 1:1:1. The two coating formulations, containing trivalent HA, were formulated with sucrose (5%), or 2) Tween-80 (2%) and sucrose (5%). The microprojection array designs were MF-1, MF-3, and MF-5 (2 cm² in diameter). The two HA coating doses loaded onto the microprojection array designs were defined as “low” (21-23 μg) and “high” (33-45 μg). The data demonstrate that trivalent Macroflux patches can induce primary anti-HA antibody responses (HI titers) to each HA strain (see FIG. 18). The antibody titer levels generated from HGPs immunized the two trivalent formulations (sucrose and Tween-80/sucrose) using Macroflux arrays were comparable to their respective intramuscular injection controls. No significant difference, with respect to anti-HA responses, was seen among the various microprojection array designs or between sucrose versus tween-80/sucrose formulations. In some cases, depending on the HA strain and treatment group, a dose response was observed but was not always the case.

Overall, these immunogenicity studies suggest that each of the formulations set forth in Table V were immunogenic despite significant formulation changes to the starting vaccine.

Short Term Stability

The pre-formulation process discussed above subjects an antigen to not only freezing, but also a series of stress events, including shear stress during membrane diafiltration, and stress arising from ice/water interface and dehydration/rehydration. After reconstituting the freeze-dried vaccine, the solution was thus subjected to 10 cycles of freeze/thaw (frozen by liquid nitrogen and immediately thawed at room temperature) to assess the effects, if any, on the stability of the antigen. As determined by ELISA, the HA potency before and after 10 cycles of freeze/thaw was unchanged, suggesting the preservation of antigen stability by the trehalose or sucrose.

An even more stressful process step than freeze-drying, is re-solubilization by a potent surfactant, such as SDS or Zwittergent at high concentrations with vigorous shaking (vortexing). These surfactants are known to denature proteins by altering the physical conformation of the native molecule. To vaccine antigens, the consequence of significant conformational changes might be total loss of antigenicity and immunogenicity. The effect of re-solubilization in the presence of strong surfactants was assessed in the following studies.

SDS-PAGE/Western blot analysis was performed on A/Panama vaccine after a series of pre-formulation steps including the freeze-dried vaccine reconstituted without surfactant and with SDS (at 10%), Triton-X 100 (at 10%), or Zwittergent 3-14 (at 5 and 10%). Under the non-reducing conditions for the Coomassie Blue stained gels (SDS-PAGE gels on the left), it was evident that all bands present in the starting vaccine were also present in the reconstituted samples, suggesting no detectable degradation for any of the formulations evaluated.

As the gel was transferred to the membrane for Western Blot analysis, again, no differences were noticed between the different formulations and the starting vaccine. A series of bands, reflecting the binding between HA protein and anti-HA antibodies, occurred primarily at high molecular weights. Based on the matched bands and band intensity (relative to the starting vaccine), it was concluded that the HA in formulations that had been freeze-dried and exposed to high concentrations of strong surfactants maintained antigenicity.

Under reducing conditions, all formulations show bands similar to that of the starting vaccine on SDS-PAGE gels. Band patterns on the Western Blot gels were also matched nicely among all formulations. Along with ELISA analysis, HA appears to be robust and remains antigenic even after extensive formulation manipulation including diafiltration, concentration, freezing, dehydration, and re-hydration with strong surfactant under intensive vortexing.

Long-Term Stability

Two types of stability were investigated to screen and identify the optimal formulation: (i) the physical stability of the coating and (ii) the biochemical stability of the antigen, both of which need to be maintained during storage to preserve the deliverable target dose.

Physical Stability

The physical stability of the coating includes the preservation of the coating's location and morphology after storage at a specific temperature for a certain period of time. To facilitate the study, four coating formulations (Nos. 3, 5, 6 and 7) were exposed to high temperature (65° C.) for up to four weeks.

The SEM morphology of Formulations 5 & 6 before and after storage at 65° C. indicated that no changes occurred upon storage for four weeks. The same result was observed for Formulations 3 & 7, suggesting that all four formulations coated are physically robust even at such high temperatures.

Biochemical Stability

Referring to Table XIII, there is a similarly of the parameters employed to investigate the antigens biochemical stability. The investigation involved four studies, which started with an accelerated study for screening Formulation Nos. 3, 5, 6 and 7 using a monovalent strain. The most stable formulation(s) were tested in an excipient dilution study with the other two strains at a series of excipient composition. The preferred composition determined from the excipient dilution study was then tested in a trivalent formulation coated onto microprojection arrays packaged in the foil pouch as part of informal stability study. This final packaged stability study was conducted to investigate the effect of moisture content in the coating on antigen stability.

TABLE XIII

| | | | Stability- |
|---|---|---|---|
| Study Type | Formulations | Conditions | Indicating Assay |
| Accelerated | A/Panama of 3, 5, | 40° C. and room | ELISA |
| | 6, 7 (Table 5) | temperature | |
| Dilution | A/New Caledonia | 40° C. and room | SRID |
| | and B/Victoria | temperature | |
| Packaging | Trivalent | 2-8, 25, and | SRID |
| | | 40° C. | |
| Moisture effect | Trivalent | 40° C. | SRID |

Accelerated Stability Study

Four A/Panama formulations (Formulation Nos. 3, 5, 6 and 7) were coated onto microprojection arrays. Each coated array was placed in a 20-mL scintillation vial with a screw top cap. Each vial was sealed after vacuum drying to remove moisture up-take following array handling. All samples were incubated in a 40° C. oven for 1, 2, 4, and 8 weeks. Three samples (triplicates) were taken at each time point and analyzed for HA potency by ELISA.

Referring now to FIG. 18, there is shown the stability profile of the four formulations. There is a clear trend for the Zwittergent formulation (Formulation No. 3) whose HA potency appeared to decrease with the incubation time. It was also evident that the Tween/sucrose formulation seemed to lose the majority of the HA potency at the final time point (Week 8).

The stability of the sucrose alone formulation was the third best of the formulations and the Pluronic/trehalose/mannitol formulation the best at maintaining potency.

Three of the noted formulations coated at two different doses were then stored at room temperature (under vacuum) for up to 25 weeks. HA potency was monitored by ELISA. Referring to Table IV, the Trehalose/Mannitol/Pluronic formulation (Formulation No. 7) showed a trend of decreasing potency. The other two formulations appeared to maintain the antigen potency, as compared to the potency at Time 0. For Formulations Nos. 5 and 7, the stability trend seemed to be different between samples stored at 40° C. and at room temperature.

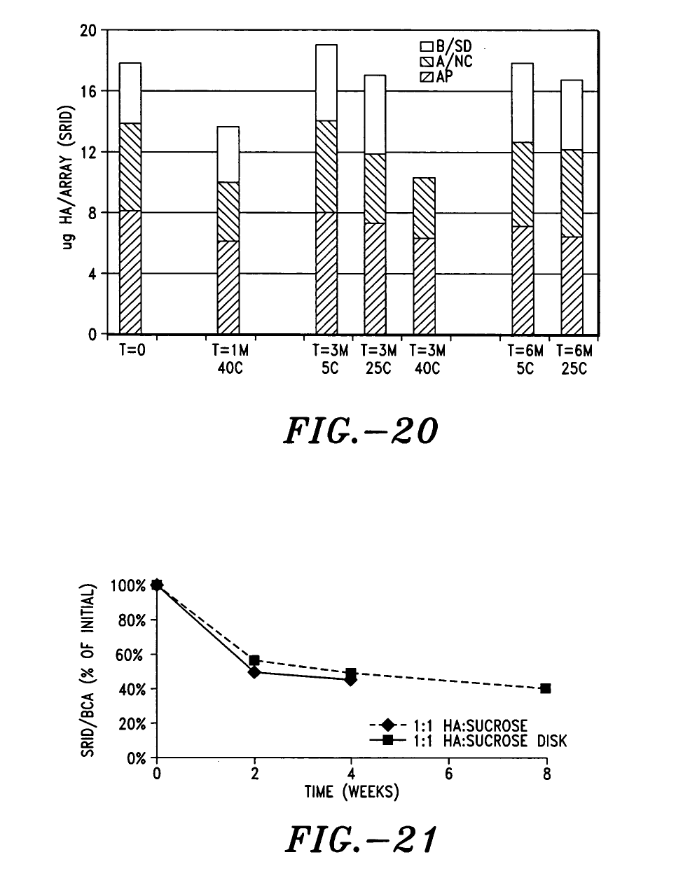

Two trivalent formulations, comprising sucrose only and sucrose-Tween, were coated on arrays and stored in sealed, nitrogen purged foil pouches for up to 3 months at 40° C. and up to 6 months at 5° and 25° C. The potency for each of the three strains A/Panama (A/P), A/New Caledonia (A/NC) and B/Shangdong (B/SD) were assayed by SRID analysis. The results of the sucrose only and sucrose-Tween formulation stability studies are presented in FIGS. 19 and 20, respectively. As reflected in FIGS. 19 and 20, the coated arrays showed very good stability for up to 6 months storage at 5° and 25° C. for all three strains in both formulations.

Excipient Dilution Study

To determine the optimal excipient composition for the sucrose formulation, the B/Victoria strain (18% of HA purity) was formulated with sucrose at the weight ratios of HA:sucrose=1: 1, 1:2, and 1:4. The coated arrays were incubated at 40° C. for up to 8 weeks. Samples were stored at −80° C. until the time of analysis and all samples were reconstituted with 1 mL of water and analyzed by SRID on a single gel and by BCA on a single 96 well plate to eliminate inter-assay variability. The stability profiles are shown in FIG. 21.

Following the initial decrease observed during the first two weeks of storage, the formulations of HA:sucrose=1 :2 & 1:4 appeared to be stable through eight weeks even at 40° C. However, for the formulation of HA: sucrose=1:1, the decreasing trend continued. It is believed that this phenomenon is caused by the presence of protease, which was not completely removed during purification and which may be activated during our process.

There appears to be a stabilizing effect with increasing amounts of sucrose relative to HA. The lot of B/Victoria used for this study had a very low HA purity, ˜15% relative to total protein present, and was not anticipated to be indicative of future bulk starting material (≧40% HA purity). The stabilizing effect of increasing the sucrose weight percentage may not however be observed to an equivalent relative degree with higher HA purity starting material. For example, 100 mg of 15% HA purity starting material requires 15, 30 and 45 mg sucrose when formulated at 1: 1, 1:2 and 1:4 HA:sucrose. This results in dry weight ratios of 13, 23 and 37% sucrose, respectively. However, 100 mg of 40% HA purity starting material would require 40, 80 and 160 mg sucrose to formulate at the same three ratios, resulting in dry weight ratios of 29, 44 and 54% sucrose. As a result, the high purity 1:1 formulation is already approaching the dry weight sucrose content of the 1:4 low purity formulation. At these levels, the stabilizing effect of sucrose has most likely reached a plateau and increasing the sucrose content any further would have little or no effect on the stability of the product. For this reason and to simplify further bulk processing, a fixed-ratio of sucrose was set at 1.0% for the pre-lyophilized solution. As the lyophilized power is typically reconstituted to ⅕ the original pre-lyophilized volume, this results in a coating solution concentration of 5% sucrose.

Summary

As will be appreciated by one having ordinary skill in the art, by virtue of the unique-preformulation process, a full human dose of the influenza vaccine, i.e., 45 μg of hemagglutinin, can be transdermally delivered via a coated microprojection array, wherein at least 70% of the influenza vaccine is delivered into the skin. The antigen also remains immunogenic in the skin to elicit strong antibody and sero-protective immune responses. Further, the dry coated vaccine formulation is substantially preservative-fee and can maintain at least a six-month room temperature stability.

Without departing from the spirit and scope of this invention, one of ordinary skill can make various changes and modifications to the invention to adapt it to various usages and conditions. As such, these changes and modifications are properly, equitably, and intended to be, within the full range of equivalence of the following claims.

1. A system for transdermally delivering an immunologically active agent, comprising a microprojection member having a plurality of stratum corneum-piercing microprojections having a biocompatible coating disposed on said microprojections, wherein said coating contains said immunologically active agent.

2. The system of claim 1, wherein said biocompatible coating is formed from a formulation of said immunologically active agent.

3. The system of claim 1, wherein said immunologically active agent comprises an influenza vaccine.

4. The system of claim 1, wherein said immunologically active agent is selected from the group consisting of viruses, bacteria, protein-based vaccines, polysaccharide-based vaccine, and nucleic acid-based vaccines.

5. The system of claim 1, wherein said immunologically active agent is selected from the group consisting of viruses, weakened viruses, killed viruses, bacteria, weakened bacteria, killed bacteria, protein-based vaccines, polysaccharide-based vaccine, nucleic acid-based vaccines, proteins, polysaccharide conjugates, oligosaccharides, lipoproteins, Bordetella pertussis (recombinant PT vaccine—acellular), Clostridium tetani (purified, recombinant), Corynebacterium diphtheriae (purified, recombinant), Cytomegalovirus (glycoprotein subunit), Group A streptococcus (glycoprotein subunit, glycoconjugate Group A polysaccharide with tetanus toxoid, M protein/peptides linked to toxing subunit carriers, M protein, multivalent type-specific epitopes, cysteine protease, C5a peptidase), Hepatitis B virus (recombinant Pre S1, Pre-S2, S, recombinant core protein), Hepatitis C virus (recombinant—expressed surface proteins and epitopes), Human papillomavirus (Capsid protein, TA-GN recombinant protein L2 and E7 [from HPV-6], MEDI-501 recombinant VLP L1 from HPV-1 1, Quadrivalent recombinant BLP L1 [from HPV-6], HPV-11, HPV-16, and HPV-18, LAMP-E7 [from HPV-16]), Legionella pneumophila (purified bacterial survace protein), Neisseria meningitides (glycoconjugate with tetanus toxoid), Pseudomonas aeruginosa (synthetic peptides), Rubella virus (synthetic peptide), Streptococcus pneumoniae (glyconconjugate [1, 4, 5, 6B, 9N, 14, 18C, 19V, 23F] conjugated to meningococcal B OMP, glycoconjugate [4, 6B, 9V, 14, 18C, 19F, 23F] conjugated to CRM197, glycoconjugate [1, 4, 5, 6B, 9V, 14, 18C, 19F, 23F] conjugated to CRM1970, Treponema pallidum (surface lipoproteins), Varicella zoster virus (subunit, glycoproteins), Vibrio cholerae (conjugate lipopolysaccharide), cytomegalo virus, hepatitis B virus, hepatitis C virus, human papillomavirus, rubella virus, varicella zoster, bordetella pertussis, clostridium tetani, corynebacterium diphtheriae, group A streptococcus, legionella pneumophila, neisseria meningitdis, pseudomonas aeruginosa, streptococcus pneumoniae, treponema pallidum, vibrio cholerae, flu vaccines, Lyme disease vaccines, rabies vaccines, measles vaccines, mumps vaccines, chicken pox vaccines, small pox vaccines, hepatitis vaccines, pertussis vaccines, diphtheria vaccines, nucleic acids, single-stranded nucleic acids, double-stranded nucleic acids, supercoiled plasmid DNA, linear plasmid DNA, cosmids, bacterial artificial chromosomes (BACs), yeast artificial chromosomes (YACs), mammalian artificial chromosomes, RNA molecules, and mRNA.

6. The system of claim 1, wherein said formulation further comprises an immune response augmenting adjuvant selected from the group consisting of aluminum phosphate gel, aluminum hydroxide, alpha glucan, β-glucan, cholera toxin B subunit, CRL1005, ABA block polymer with mean values of x=8 and y=205, gamma inulin, linear (unbranched) β-D(2->1) polyfructofuranoxyl-α-D-glucose, Gerbu adjuvan, N-acetylglucosamine-(β 1-4)-N-acetylmuramyl-L-alanyl-D-glutamine (GMDP), dimethyl dioctadecylammonium chloride (DDA), zinc L-proline salt complex (Zn-Pro-8), Imiquimod (1-(2-methypropyl)-1H-imidazo[4,5-c]quinolin-4-amine, ImmTher™, N-acetylglucoaminyl-N-acetylmuramyl-L-Ala-D-isoGlu-L-Ala-glycerol dipalmitate, MTP-PE liposomes, C₅₉H₁₀₈N₆O₁₉PNa-3H₂O (MTP), Murametide, Nac-Mur-L-Ala-D-Gln-OCH₃, Pleuran, QS-21; S-28463, 4-amino-a, a-dimethyl-1H-imidazo[4,5-c]quinoline-1-ethanol, sclavo peptide, VQGEESNDK•HCl (IL-1β 163-171 peptide), threonyl-MDP (Termurtide™), N-acetyl muramyl-L-threonyl-D-isoglutamine, interleukine 18 (IL-18), IL-2 IL-12, IL-15, IL-4, IL-10, DNA oligonucleotides, CpG containing oligonucleotides, gamma interferon, and NF kappa B regulatory signaling proteins.

7. The system of claim 1, wherein said microprojection member has a microprojection density of at least approximately 100 microprojections/cm².

8. The system of claim 7, wherein said microprojection member has a microprojection density in the range of approximately 200-3000 microprojections/cm².

9. The system of claim 1, wherein each of said microprojections has a length in the range of approximately 50-145 microns.

10. The system of claim 9, wherein each of said microprojections has a length in the range of approximately 70-140 microns.

11. The system of claim 1, wherein said biocompatible coating has a thickness in the range of approximately 2-50 microns.

12. The system of claim 2, wherein said formulation comprises an aqueous formulation.

13. The system of claim 2, wherein said coating formulation includes a surfactant.

14. The system of claim 13, wherein said surfactant is selected from the group consisting of sodium lauroamphoacetate, sodium dodecyl sulfate (SDS), cetylpyridinium chloride (CPC), dodecyltrimethyl ammonium chloride (TMAC), benzalkonium, chloride, polysorbates, such as Tween 20 and Tween 80, sorbitan derivatives, sorbitan laurate, alkoxylated alcohols, and laureth-4.

15. The system of claim 2, wherein said coating formulation includes an amphiphilic polymer.

16. The system of claim 15, wherein said amphiphilic polymer is selected from the group consisting of cellulose derivatives, hydroxyethylcellulose (HEC), hydroxypropyl-methylcellulose (HPMC), hydroxypropycellulose (HPC), methylcellulose (MC), hydroxyethylmethylcellulose (HEMC), ethylhydroxyethylcellulose (EHEC), and pluronics.

17. The system of claim 2, wherein said coating formulation includes a hydrophilic polymer.

18. The system of claim 17, wherein said hydrophilic polymer is selected from the group consisting of poly(vinyl alcohol), poly(ethylene oxide), poly(2-hydroxyethylmethacrylate), poly(n-vinyl pyrolidone), polyethylene glycol and mixtures thereof.

19. The system of claim 2, wherein said coating formulation includes a biocompatible carrier.

20. The system of claim 19, wherein said biocompatible polymer is selected from the group consisting of human albumin, bioengineered human albumin, polyglutamic acid, polyaspartic acid, polyhistidine, pentosan polysulfate, polyamino acids, sucrose, trehalose, melezitose, raffinose and stachyose.

21. The system of claim 2, wherein said coating formulation includes a stabilizing agent selected from the group consisting of a non-reducing sugar, a polysaccharide, a reducing sugar, and a DNase inhibitor.

22. The system of claim 2, wherein said coating formulation includes a vasoconstrictor.

23. The system of claim 22, wherein said vasoconstrictor is selected from the group consisting of epinephrine, naphazoline, tetrahydrozoline indanazoline, metizoline, tramazoline, tymazoline, oxymetazoline, xylometazoline, amidephrine, cafaminol, cyclopentamine, deoxyepinephrine, epinephrine, felypressin, indanazoline, metizoline, midodrine, naphazoline, nordefrin, octodrine, ornipressin, oxymethazoline, phenylephrine, phenylethanolamine, phenylpropanolamine, propylhexedrine, pseudoephedrine, tetrahydrozoline, tramazoline, tuaminoheptane, tymazoline, vasopressin and xylometazoline.

24. The system of claim 2, wherein said coating formulation includes a pathway patency modulator.

25. The system of claim 24, wherein said pathway patency modulator is selected from the group consisting of osmotic agents, sodium chloride, zwitterionic compounds, amino acids, anti-inflammatory agents, betamethasone 21-phosphate disodium salt, triamcinolone acetonide 21-disodium phosphate, hydrocortamate hydrochloride, hydrocortisone 21-phosphate disodium salt, methylprednisolone 21-phosphate disodium salt, methylprednisolone 21-succinaate sodium salt, paramethasone disodium phosphate, prednisolone 21-succinate sodium salt, anticoagulants, citric acid, citrate salts, sodium citrate, dextran sulfate sodium, and EDTA.

26. The system of claim 2, wherein said coating formulation has a viscosity less than approximately 5 poise and greater than approximately 0.3 poise.

27. A method for transdermally delivering an immunologically active agent to a subject, the method comprising the steps of:
providing a microprojection member having a plurality of microprojections;
providing a bulk vaccine;
subjecting said bulk vaccine to tangential-flow filtration to provide a vaccine solution;
adding at least one excipient to said vaccine solution;
freeze-drying said vaccine solution to form a vaccine product;
reconstituting said vaccine product with a first solution to form a vaccine coating formulation;
coating said microprojection member with said vaccine coating formulation; and
applying said coated microprojection member to the skin of said subject.

28. The method of claim 27, wherein the step of adding at least one excipient to said vaccine solution comprises adding an excipient selected from the group consisting of sucrose, trehalose and mannitol.

29. The method of claim 27, wherein the step of providing a bulk vaccine comprises providing an influenza vaccine.

30. The method of claim 29, wherein said influenza vaccine comprises hemagluttinin.

31. The method of claim 30, further comprising the step of delivering approximately 45 μg of hemagglutinin.

32. The method of claim 31, wherein the step of delivering hemagglutinin comprises delivering at least approximately 70% of said hemagglutinin to an APC-abundant layer of said subject's epidermis.

33. A method for formulating a vaccine solution comprising the steps of:
providing a bulk vaccine;
subjecting said bulk vaccine to tangential-flow filtration to provide a vaccine solution;
adding at least one excipient to said vaccine solution; and
freeze-drying said vaccine solution to form a vaccine product.

34. The method of claim 33, wherein said vaccine product exhibits a concentration that is at least approximately 500-fold more concentrated than said bulk vaccine.

35. The method of claim 33, wherein said vaccine product maintains room temperature stability for at least approximately six months.

36. The method of claim 33, wherein the step of providing a bulk vaccine comprises providing an influenza vaccine.

37. The method of claim 36, wherein said influenza vaccine comprises hemagluttinin.
